(19) United States
(12) Patent Application Publication
(10) Pub. No.: US 2025/0152557 A1
(43) Pub. Date: May 15, 2025

(54) METHODS FOR TREATING SUBJECTS WITH ABDOMINAL OBESITY HYPERTRIGLYCERIDEMIA AND/OR IMPAIRED GLUCOSE

(71) Applicant: NOVO NORDISK A/S, Bagsvaerd (DK)

(72) Inventors: Glenn Crater, Raleigh, NC (US); Francois Ravenelle, Montreeal, CA (US); Michael Harvey, Montreal (CA)

(21) Appl. No.: 18/839,945

(22) PCT Filed: Feb. 24, 2023
(86) PCT No.: PCT/CA2023/050236

§ 371 Date: Aug. 20, 2024

Related U.S. Application Data
(60) Provisional application No. 63/268,460, filed on Feb. 24, 2022.

(51) Int. Cl. A61K 31/415; A61P 3/04

(52) U.S. Cl. CPC A61K 31/415; A61P 3/04

(57) ABSTRACT

The present description relates to a CB1 receptor inhibitor compound of Formula I or a pharmaceutically acceptable salt thereof for the treatment of a patient population with abdominal obesity hypertriglyceridemia and impaired glucose.

### BACKGROUND

It is generally known that activation of the cannabinoid CB₁ receptor increases appetite, increases the biosynthesis and storage of lipids, inhibits the actions of insulin and leptin, and promotes inflammation and fibrosis. Research was thus focused on developing CB₁ receptor inhibitors for the potential treatment of obesity and the metabolic disorder associated therewith, referred to as metabolic syndrome. Rimonabant was shown effective in treating metabolic syndrome but caused neuropsychiatric (i.e. CNS-related) side effects, which resulted in its withdrawal from the market.

Compounds preferentially targeting the CB₁ receptor in peripheral tissues (e.g. adipose tissue, liver, muscle, lung, kidney, macrophages, pancreatic beta cells and gastrointestinal tract), were disclosed by George Kunos et al. in U.S. Pat. No. 9,765,031.

One of the compounds is N-{[(S)-{[3-(4-chlorophenyl)-4-phenyl-4,5-dihydro-1H-pyrazol-1-yl][4-(trifluoromethyl)benzenesulfonamido]methylidene}amino]methanimidoyl}acetamide also referred to as INV-202.

### SUMMARY

In some embodiments, the present disclosure pertains to uses and methods for treating a patient population having abdominal obesity. It was surprisingly found that a daily oral administration of the compound of formula I in a patient population having abdominal obesity, hypertriglyceridemia and impaired glucose resulted in a marked weight loss combined with a significant reduction in triglycerides without the patients experiencing non-specific suicidal thoughts during treatment. A statistically significant weight loss was noted after only 8 days of treatment.

In some embodiments, the present disclosure pertains to the use of a compound of Formula I or a pharmaceutically acceptable salt thereof:

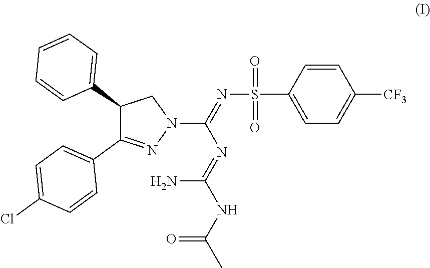 [SMILES: CC(=O)N/C(N)=N/C(=N\S(=O)(=O)c1ccc(C(F)(F)F)cc1)N1C[C@H](c2ccccc2)C(c2ccc(Cl)cc2)=N1]

to reduce body weight and triglycerides in a human subject determined as having abdominal obesity characterized by a waist circumference ≥88 cm for female subjects or ≥102 cm for male subjects, wherein said reduction is relative to baseline level before treatment and wherein the compound of Formula I is for oral administration to the human subject for a period of at least 28 days.

In some embodiments, the present disclosure pertains to the use of a compound of Formula I or a pharmaceutically acceptable salt thereof to reduce body weight, triglycerides and glucose intolerance in a human subject determined as having abdominal obesity characterized by a waist circumference ≥88 cm for female subjects or ≥102 cm for male subjects, wherein said reduction is relative to baseline level before treatment and wherein the compound of Formula I is for oral administration to the human subject for a period of at least 28 days.

In some embodiments, the disclosure pertains to a compound of Formula I or a pharmaceutically acceptable salt thereof for use in reducing body weight and triglycerides in a human subject determined as having abdominal obesity characterized by a waist circumference ≥88 cm for female subjects or 102 cm for male subjects, wherein said reduction is relative to baseline level before treatment and wherein the compound of Formula I is for oral administration to the human subject for a period of at least 28 days.

In some embodiments, the disclosure pertains to a compound of Formula I or a pharmaceutically acceptable salt thereof for use in reducing body weight, triglycerides and glucose intolerance in a human subject determined as having abdominal obesity characterized by a waist circumference ≥88 cm for female subjects or 102 cm for male subjects, wherein said reduction is relative to baseline level before treatment and wherein the compound of Formula I is for oral administration to the human subject for a period of at least 28 days.

In some embodiments, the disclosure pertains to a method for reducing body weight and triglycerides in a human subject determined as having abdominal obesity characterized by a waist circumference ≥88 cm for female subjects or 102 cm for male subjects, wherein said reduction is relative to baseline level before treatment said method comprising orally administering a compound of Formula I or a pharmaceutically acceptable salt thereof to the human subject for a period of at least 28 days.

In some embodiments, the disclosure pertains to a method for reducing body weight, triglycerides and glucose intolerance in a human subject determined as having abdominal obesity characterized by a waist circumference ≥88 cm for female subjects or ≥102 cm for male subjects, wherein said reduction is relative to baseline level before treatment said method comprising orally administering a compound of Formula I or a pharmaceutically acceptable salt thereof to the human subject for a period of at least 28 days.

In some embodiments, the disclosure pertains to a method for reducing subjective appetite sensations in a subject in a subject in need thereof, said method comprising administering a compound of Formula I or a pharmaceutically acceptable salt thereof to the subject, wherein said reducing is relative to baseline level.

In some embodiments, the reduction of subjective appetite sensations is determined by an Appetite and Food Intake Questionnaire

In some embodiments, the subject is a human subject and presents one or more of the following:
waist circumference ≥88 cm for female subjects or ≥102 cm for male subjects;
fasting triglyceride (e.g. >1.5 mmol/L for males and females) and/or
impaired glucose tolerance (e.g. an OGTT indicating impaired glucose tolerance as indicated by a 2-hour value >140 mg/dl or any value >200 mg/dl at any time point or a HbA1C level ≥5.7% but ≤6.4%.)

In some embodiments, the compound of Formula I is for oral administration to the subject.

In some embodiments, the compound of Formula I is for oral administration to the subject at a dose of 25 mg or more.

In some embodiments, the compound of Formula I is for daily oral administration to the subject at a dose of 25 mg or more.

In some embodiments, compound of Formula I is for daily oral administration to the subject at a dose of about 25 mg.

In some embodiments, the oral dosage form is a tablet, a capsule, a lozenge, a pastille or a granule.

In some embodiments, compound of Formula I is for daily oral administration in a unit dosage form.

In some embodiments, the pharmaceutical composition is formulated as an oral suspension.

In some embodiments, the subject exhibits no significant changes relative to baseline for bilirubin, AST and/or ALT after 28 days of treatment with the compound of formula I.

In some embodiments, the subject exhibits a body weight reduction relative to baseline between about 0.1 and about 7%, between about 1 and about 6%, between about 2 and about 5%, between about 3 and about 5%, about 4%, about 3.5%, about 3% or about 2.5%, about 2%, about 1%, about 0.5%, about 0.1% after 28 days of treatment with the compound of formula I.

In some embodiments, the subject exhibits a triglycerides reduction relative to baseline between about 1 and about 10%, between about 2 and about 9%, between about 3 and about 8%, about 8%, about 7%, about 6% or about 5% after 28 days of treatment with the compound of formula I.

In some embodiments, the subject exhibits a change in leptin levels relative to baseline of about between 10% and about 20%, of about 10%, about 15%, about 16% m about 17%, about 18%, about 19% or about 20% after 28 days of treatment with the compound of formula I.

In some embodiments, the subject exhibits a reduction in LDL level relative to baseline of at least about 0.1 mmol/L to about 1 mmol/L after 28 days of treatment with the compound of formula I.

In another aspect, there is provided the use of the compound of Formula I as described herein or the pharmaceutical composition as described herein, for the treatment of a disease or disorder selected from the group consisting of: obesity (type I or II), non-alcoholic and alcoholic fatty liver disease (a risk factor for insulin resistance), a co-morbidity of obesity, a co-morbidity of diabetes, Prader-Willi Syndrome (PWS), Pro-opiomelanocortin (POMC) deficiency obesity, LepR deficiency obesity, POMC heterozygous deficiency obesity, POMC epigenetic disorders, Bardet-Biedl syndrome, Alström syndrome, dyslipidemia predisposing to arteriosclerotic heart disease, diabetic nephropathy, fibrosis and fibrotic diseases such as Idiopathic Pulmonary Fibrosis (IPF) and Hermansky-Pudlak Syndrome pulmonary fibrosis (HPS-PF), gout, Primary Biliary Cirrhosis (PBC) and Primary Sclerosing Cholangitis (PSC).

In another aspect, there is provided the use of the compound of Formula I as described herein or the pharmaceutical composition as described herein, for the manufacture of a medicament for the treatment of a disease or disorder selected from the group consisting of: obesity (type I or II), non-alcoholic and alcoholic fatty liver disease (a risk factor for insulin resistance), a co-morbidity of obesity, a co-morbidity of diabetes, Prader-Willi Syndrome (PWS), Pro-opiomelanocortin (POMC) deficiency obesity, LepR deficiency obesity, POMC heterozygous deficiency obesity, POMC epigenetic disorders, Bardet-Biedl syndrome, Alström syndrome, dyslipidemia predisposing to arteriosclerotic heart disease, diabetic nephropathy, fibrosis and fibrotic diseases such as Idiopathic Pulmonary Fibrosis (IPF) and Hermansky-Pudlak Syndrome pulmonary fibrosis (HPS-PF), and gout.

In another aspect, there is provided a method for the treatment of a disease or disorder selected from the group consisting of obesity, diabetes (type I or II), non-alcoholic and alcoholic fatty liver disease (a risk factor for insulin resistance), a co-morbidity of obesity, a co-morbidity of diabetes, Prader-Willi Syndrome (PWS), Pro-opiomelanocortin (POMC) deficiency obesity, LepR deficiency obesity, POMC heterozygous deficiency obesity, POMC epigenetic disorders, Bardet-Biedl syndrome, Alström syndrome, dyslipidemia predisposing to arteriosclerotic heart disease, diabetic nephropathy, fibrosis and fibrotic diseases such as Idiopathic Pulmonary Fibrosis (IPF) and Hermansky-Pudlak Syndrome pulmonary fibrosis (HPS-PF), and gout, comprising administering the compound of Formula I as defined herein or the pharmaceutical composition as defined herein, to a subject in need thereof.

In some embodiments, the co-morbidity of obesity is selected from metabolic syndrome, dementia, heart disease, hypertension, gallbladder disease, gastrointestinal disorders, menstrual irregularities, degenerative arthritis, venous statis ulcer, pulmonary hypoventilation syndrome, sleep apnea, snoring, coronary artery disease, arterial sclerotic disease, pseudotumor cerebri, osteoarthritis, high cholesterol, and increased incidence of malignancies of the liver, ovaries, cervix, uterus, breasts, prostate, or gallbladder.

In some embodiments, the co-morbidity of diabetes (e.g. type I) is selected from diabetic nephropathy, chronic kidney disease, diabetic retinopathy, and peripheral and autonomic neuropathy.

In some embodiments, the disease or disorder is selected from diabetes (type 1 or 2), obesity, and non-alcoholic fatty liver disease (e.g. non-alcoholic steatohepatitis).

### BRIEF DESCRIPTION OF THE FIGURES

FIG. 1 shows the weight difference (kg) between INV-202 (full bars) and placebo (stripped bars).

### DETAILED DESCRIPTION

### Definitions

The terminology used herein is for the purpose of describing particular embodiments only and is not intended to be limiting. It should be noted that, the singular forms “a”, “an”, and “the” include plural forms as well, unless the content clearly dictates otherwise. Thus, for example, reference to a composition containing “a compound” also contemplates a mixture of two or more compounds. It should also be noted that the term “or” is generally employed in its sense including “and/or” unless the context clearly dictates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

The present description provides the compound of Formula I or a pharmaceutically acceptable salt thereof:

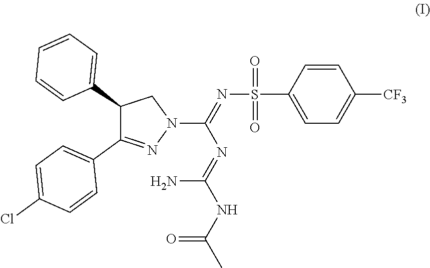 [SMILES: CC(=O)N/C(N)=N/C(=N\S(=O)(=O)c1ccc(C(F)(F)F)cc1)N1C[C@H](c2ccccc2)C(c2ccc(Cl)cc2)=N1]

The structure depicted for the compound of Formula I is also meant to include all tautomeric forms of the compound of Formula I. Additionally structures depicted herein are also meant to include compounds that differ only in the presence of one or more isotopically enriched atoms. For example, compounds having the structure of the compound of Formula I except for the replacement of hydrogen by deuterium or tritium, or the replacement of a carbon by a ¹³C- or ¹⁴C-enriched carbon are within the scope of the present description. The compound of Formula (I) is also referred to as INV-202.

### Formulations, Methods and Uses

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As used herein, the terms “reduce”, “reducing”, “improve”, “improving”, “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

The term “patient or subject” as used herein refers to a mammal. A subject therefore refers to, for example, dogs, cats, horses, cows, pigs, guinea pigs, and the like. Preferably the subject is a human. When the subject is a human, the subject may be either a patient or a healthy human. In one aspect, the subject is a human subject having metabolic syndrome. In one aspect, the subject is a human subject having abdominal obesity.

The term “metabolic syndrome” as used herein refers to a subject having hypertriglyceridemia, abdominal obesity, and impaired glucose tolerance.

The term “glucose intolerance” as used herein refers to an impaired glucose tolerance as determined by an oral glucose tolerance test (OGTT) with 75 g glucose load, by a 2-hour value >140 mg/dl or any value >200 mg/dl at any time point, or a HbA1C level ≥5.7% but ≤6.4%.

The term “baseline level” as used herein refers to the value before the subject begins treatment with a compound of the formula (I). In some instance, the baseline value can also be the normal value for a comparable reference group.

The term “plasma lipid profile” as used herein refers to the levels of total cholesterol, high-density lipoprotein (HDL), low-density lipoprotein (LDL), very-low-density lipoprotein (VLDL), and triglycerides.

The term “subjective appetite sensations” as used herein refers to is determined by an Appetite and Food Intake Questionnaire as depicted herein taken before, during and after treatment.

The present description provides a method of treating a disorder (as described herein) in a subject, comprising administering to the subject identified as in need thereof, the compound of Formula I. The identification of those patients who are in need of treatment for the disorders described above is well within the ability and knowledge of one skilled in the art. Certain of the methods for identification of patients which are at risk of developing the above disorders which can be treated by the subject method are appreciated in the medical arts, such as family history, and the presence of risk factors associated with the development of that disease state in the subject patient. A clinician skilled in the art can readily identify such candidate patients, by the use of, for example, clinical tests, physical examination, medical/family history, and genetic determination.

A method of assessing the efficacy of a treatment in a subject includes determining the pre-treatment symptoms of a disorder by methods well known in the art and then administering a therapeutically effective amount of a compound of the present description, to the subject. After an appropriate period of time following the administration of the compound (e.g., 1 week, 2 weeks, one month, six months), the symptoms of the disorder are determined again. The modulation (e.g., decrease) of symptoms and/or of a biomarker of the disorder indicates efficacy of the treatment. The symptoms and/or biomarker of the disorder may be determined periodically throughout treatment. For example, the symptoms and/or biomarker of the disorder may be checked every few days, weeks or months to assess the further efficacy of the treatment. A decrease in symptoms and/or biomarker of the disorder indicates that the treatment is efficacious.

In some embodiment, the compounds of Formula I is for administration to the human subject for a period of at least 8 days to 35 days, at least 8 days, at least 15 days, at least 22 days, at least 28 days, at least 29 days or at least 35 days.

In some embodiment, the compounds of Formula I is for oral administration to the human subject for a period of at least 8 days to 35 days, at least 8 days, at least 15 days, at least 22 days, at least 28 days, at least 29 days or at least 35 days.

Compositions described herein may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a provided compound, it may be desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming micro-encapsulated matrices of the compound in biodegradable polymers such as polylactide-polyglycolide. Depending upon the ratio of compound to polymer and the nature of the particular polymer employed, the rate of compound release can be controlled.

Examples of other biodegradable polymers include poly(orthoesters) and poly-(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of the present description with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents. In one aspect, the compound of Formula I is formulated as a solid dispersion as described in WO2021189141A1.

Solid compositions of a similar form may also be employed as fillers in soft and hard-filled gelatin capsules using such excipients as lactose or milk sugar as well as high molecular weight polyethylene glycols and the like. The solid dosage forms of tablets, dragees, lozenges, capsules, pastilles, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar Form may also be employed as fillers in soft and hard-filled gelatin capsules using such excipients as lactose or milk sugar as well as high molecular weight polyethylene glycols and the like.

Provided compounds can also be in micro-encapsulated form with one or more excipients as noted above. The solid dosage forms of tablets, dragees, lozenges, capsules, pastilles, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of the present description include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of the present description. Additionally, the description contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Pharmaceutically acceptable compositions provided herein may also be administered by nasal aerosol or inhalation. Such compositions are prepared according to techniques well-known in the art of pharmaceutical formulation and may be prepared as solutions in saline, employing benzyl alcohol or other suitable preservatives, absorption promotors to enhance bioavailability, fluorocarbons, and/or other conventional solubilizing or dispersing agents.

Pharmaceutically acceptable compositions provided herein may be formulated for oral administration. Such formulations may be administered with or without food. In some embodiments, pharmaceutically acceptable compositions of this disclosure are administered without food. In other embodiments, pharmaceutically acceptable compositions of this disclosure are administered with food.

Pharmaceutically acceptable compositions provided herein may be formulated for oral administration. Such formulations may be administered with or without food. The compositions are formulated in unit dosage forma for ease of administration and uniformity of dosage. The expression “unit dosage form” as used herein refers to a physically discrete unit of agent appropriate for the patient to be treated. It will be understood, however, that the total daily usage of the compositions of the present disclosure will be decided by the attending physician within the scope of sound medical judgment.

The amount of the compound of Formula I that may be included in a single dosage form will vary depending upon the patient to be treated (e.g. child vs adult, etc). Treatment regimens may comprise administration to a patient a total amount of from about 10 mg to about 200 mg, 25 mg to 150 mg, 25 mg to 125 mg, 25 mg to 100 mg, 25 mg to 75 mg, 25 mg to 50 mg, about 25 mg, about 50 mg of the compound of the present description per day in a single dose or divided in multiple doses.

It will be understood, that the total daily dose of the compound of Formula I will be decided by the attending physician within the scope of sound medical judgment. For instance, a specific dosage or treatment regimen for any particular patient will depend upon a variety of factors, including age, body weight, general health, sex, diet, time of administration, rate of excretion, drug combination, the judgment of the treating physician, and the severity of the symptoms associated with the disease or disorder.

Depending upon the disease or disorder to be treated, additional therapeutic agents may also be present in the compositions of this disclosure or co-administered separately. Non-limiting examples of additional therapeutic agents which could be used in combination with the compound of Formula I include antidiabetic agents, cholesterol-lowering agents, anti-inflammatory agents, antimicrobial agents, matrix metalloproteinase inhibitors, lipoxygenase inhibitors, cytokine antagonists, immunosuppressants, anti-cancer agents, anti-viral agents, cytokines, growth factors, immunomodulators, prostaglandins, or anti-vascular hyperproliferation compound. The treatment may also be complemented with other treatments or interventions such as surgery, radiotherapy (e.g., gamma-radiation, neutron beam radiotherapy, electron beam radiotherapy, proton therapy, brachytherapy, and systemic radioactive isotopes), a biologic response modifier (e.g., an interferon, an interleukin, tumor necrosis factor (TNF)), and agents used to attenuate an adverse effect of the present compound or of a co-administered ingredient.

The recitation of an embodiment for a variable herein includes that embodiment as any single embodiment or in combination with any other embodiments or portions thereof. The recitation of an embodiment herein includes that embodiment as any single embodiment or in combination with any other embodiments or portions thereof.

In some embodiments, the therapeutically effective amount of a compound as defined herein, or a pharmaceutically acceptable salt thereof, can be administered to a patient alone or admixed with a pharmaceutically acceptable carrier.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this disclosure include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of the present description that, upon administration to a recipient, is capable of providing, either directly or indirectly, a compound of the present description or an inhibitory active metabolite or residue thereof.

### Embodiments

Embodiment 1. Use of a compound of Formula I or a pharmaceutically acceptable salt thereof:

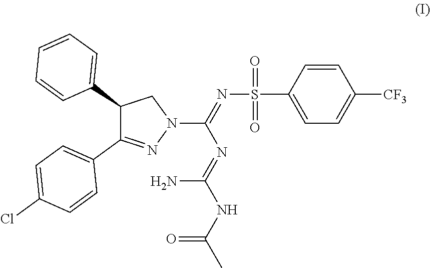 [SMILES: CC(=O)N/C(N)=N/C(=N\S(=O)(=O)c1ccc(C(F)(F)F)cc1)N1C[C@H](c2ccccc2)C(c2ccc(Cl)cc2)=N1]

to reduce body weight and triglycerides in a human subject determined as having abdominal obesity characterized by a waist circumference ≥88 cm for female subjects or 102 cm for male subjects, wherein said reduction is relative to baseline level before treatment and wherein the compound of Formula I is for oral administration to the human subject for a period of at least 28 days.
Embodiment 2. Use of a compound of Formula I or a pharmaceutically acceptable salt thereof:

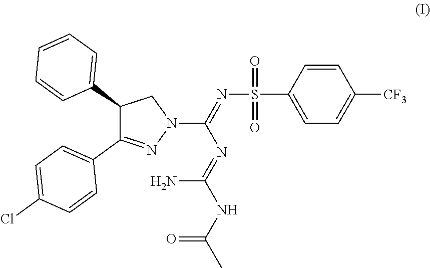 [SMILES: CC(=O)N/C(N)=N/C(=N\S(=O)(=O)c1ccc(C(F)(F)F)cc1)N1C[C@H](c2ccccc2)C(c2ccc(Cl)cc2)=N1]

ⁱⁱto reduce body weight, triglycerides and glucose intolerance in a human subject determined as having abdominal obesity characterized by a waist circumference ≥88 cm for female subjects or ≥102 cm for male subjects, wherein said reduction is relative to baseline level before treatment and wherein the compound of Formula I is for oral administration to the human subject for a period of at least 28 days.
Embodiment 3. The use of any of the preceding embodiments, wherein the subject presents one or more of the following:
   a. fasting triglycerides >1.5 mmol/L for males and females and/or
   b. an OGTT indicating impaired glucose tolerance as indicated by a 2-hour value >140 mg/dl or any value >200 mg/dl at any time point or a HbA1C level ≥5.7% but ≤6.4%.
Embodiment 4. The use of any of the preceding embodiments, wherein the subject exhibits no significant changes relative to baseline for bilirubin, AST and/or ALT after 28 days of treatment with the compound of formula I.
Embodiment 5. The use of any of the preceding embodiments, wherein the subject exhibits a body weight reduction relative to baseline between about 0.1 and about 7%, between about 1 and about 6%, between about 2 and about 5%, between about 3 and about 5%, about 4%, about 3.5%, about 3% or about 2.5% after 28 days of treatment with the compound of formula I.
Embodiment 6. The use of any of the preceding embodiments, wherein the subject exhibits a triglycerides reduction relative to baseline between about 1 and about 10%, between about 2 and about 9%, between about 3 and about 8%, about 8%, about 7%, about 6% or about 5% after 28 days of treatment with the compound of formula I.
Embodiment 7. The use of any of the preceding embodiments, wherein the subject exhibits a change in leptin levels relative to baseline of about between 10% and about 20%, after 28 days of treatment with the compound of formula I.
Embodiment 8. The use of any of the preceding embodiments, wherein the subject exhibits a reduction in LDL level relative to baseline of at least about 0.1 mmol/L to about 1 mmol/L after 28 days of treatment with the compound of formula I.
Embodiment 9. The use of any of the preceding embodiments, wherein the subject exhibits a reduction in VLDL level relative to baseline of at least about 0.03 mmol/L to about 1 mmol/L after 28 days of treatment with the compound of formula I.
Embodiment 10. The use of any of the preceding embodiments, wherein the subject exhibits a reduction in total cholesterol levels relative to baseline of at least about 0.1 mmol/L to about 1 mmol/L after 28 days of treatment with the compound of formula I.
Embodiment 11. The use of any of the preceding embodiments, wherein the subject exhibits no significant changes in subjective appetite sensations during treatment as determined by an appetite and Food Intake Questionnaire.
Embodiment 12. The use of any of the preceding embodiments, wherein the compound of Formula I is for oral administration to the subject at a dose of 25 mg or more.
Embodiment 13. The use of any of the preceding embodiments, wherein the compound of Formula I is for daily oral administration to the subject at a dose of 25 mg or more.
Embodiment 14. The use of any of the preceding embodiments, wherein the compound of Formula I is for daily oral administration to the subject at a dose of about 25 mg.
Embodiment 15. A compound of Formula I or a pharmaceutically acceptable salt thereof:

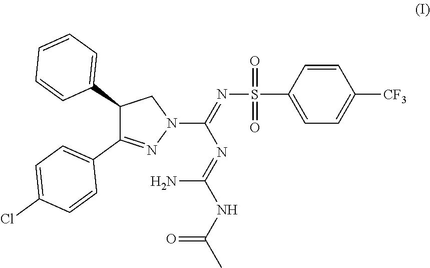 [SMILES: CC(=O)N/C(N)=N/C(=N\S(=O)(=O)c1ccc(C(F)(F)F)cc1)N1C[C@H](c2ccccc2)C(c2ccc(Cl)cc2)=N1]

for use in reducing body weight and triglycerides in a human subject determined as having abdominal obesity characterized by a waist circumference ≥88 cm for female subjects or 102 cm for male subjects, wherein said reduction is relative to baseline level before treatment and wherein the compound of Formula I is for oral administration to the human subject for a period of at least 28 days.
Embodiment 16. A compound of Formula I or a pharmaceutically acceptable salt thereof:

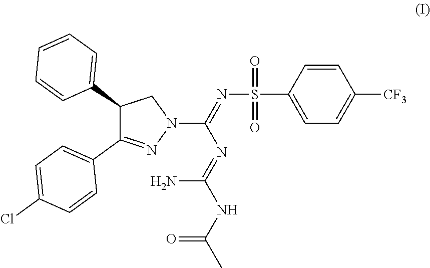 [SMILES: CC(=O)N/C(N)=N/C(=N\S(=O)(=O)c1ccc(C(F)(F)F)cc1)N1C[C@H](c2ccccc2)C(c2ccc(Cl)cc2)=N1]

for use in reducing body weight, triglycerides and glucose intolerance in a human subject determined as having abdominal obesity characterized by a waist circumference ≥88 cm for female subjects or ≥102 cm for male subjects, wherein said reduction is relative to baseline level before treatment and wherein the compound of Formula I is for oral administration to the human subject for a period of at least 28 days.
Embodiment 17. The compound for use any of the preceding embodiments, wherein the subject presents one or more of the following:
   i. fasting triglycerides >1.5 mmol/L for males and females and/or
   ii. an OGTT indicating impaired glucose tolerance as indicated by a 2-hour value >140 mg/dl or any value >200 mg/dl at any time point or a HbA1C level ≥5.7% but ≤6.4%.
Embodiment 18. The compound for use of the preceding embodiments, wherein the subject exhibits no significant changes relative to baseline for bilirubin, AST and/or ALT after 28 days of treatment with the compound of formula I.
Embodiment 19. The compound for use of any of the preceding embodiments, wherein the subject exhibits body weight reduction relative to baseline between about 0.1 and about 7%, between about 1 and about 6%, between about 2 and about 5%, between about 3 and about 5%, about 4%, about 3.5%, about 3% or about 2.5% after 28 days of treatment with the compound of formula I.
Embodiment 20. The compound for use of any of the preceding embodiments, wherein the subject exhibits a triglycerides reduction relative to baseline between about 1 and about 10%, between about 2 and about 9%, between about 3 and about 8%, about 8%, about 7%, about 6% or about 5% after 28 days of treatment with the compound of formula I.
Embodiment 21. The compound for use of any of the preceding embodiments, wherein the subject exhibits a change in leptin levels relative to baseline of about between 10% and about 20%, after 28 days of treatment with the compound of formula I.
Embodiment 22. The compound for use of any of the preceding embodiments, wherein the subject exhibits a reduction in LDL level relative to baseline of at least about 0.1 mmol/L to about 1 mmol/L after 28 days of treatment with the compound of formula I.
Embodiment 23. The compound for use of any of the preceding embodiments wherein the subject exhibits a reduction in VLDL level relative to baseline of at least about 0.03 mmol/L to about 1 mmol/L after 28 days of treatment with the compound of formula I.
Embodiment 24. The compound for use of any of the preceding embodiments, wherein the subject exhibits a reduction in total cholesterol levels relative to baseline of at least about 0.1 mmol/L to about 1 mmol/L after 28 days of treatment with the compound of formula I.
Embodiment 25. The compound for use of any of the preceding embodiments, wherein the compound of Formula I is for oral administration to the subject at a dose of 25 mg or more.
Embodiment 26. The compound for use of any of the preceding embodiments, wherein the compound of Formula I is for daily oral administration to the subject at a dose of 25 mg or more.
Embodiment 27. The compound for use of any of the preceding embodiments, wherein the compound of Formula I is for daily oral administration to the subject at a dose of about 25 mg.
Embodiment 28. A method for reducing body weight and triglycerides in a human subject determined as having abdominal obesity characterized by a waist circumference ≥88 cm for female subjects or 102 cm for male subjects, wherein said reduction is relative to baseline level before treatment said method comprising orally administering a compound of Formula I or a pharmaceutically acceptable salt thereof:

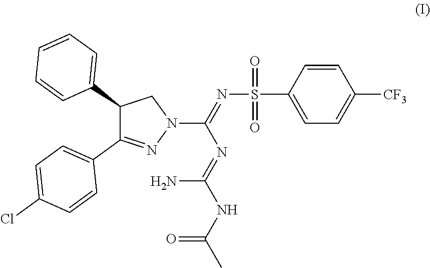 [SMILES: CC(=O)N/C(N)=N/C(=N\S(=O)(=O)c1ccc(C(F)(F)F)cc1)N1C[C@H](c2ccccc2)C(c2ccc(Cl)cc2)=N1]

to the human subject for a period of at least 28 days.
Embodiment 29. A method for reducing body weight, triglycerides and glucose intolerance in a human subject determined as having abdominal obesity characterized by a waist circumference ≥88 cm for female subjects or 102 cm for male subjects, wherein said reduction is relative to baseline level before treatment said method comprising orally administering a compound of Formula I or a pharmaceutically acceptable salt thereof:

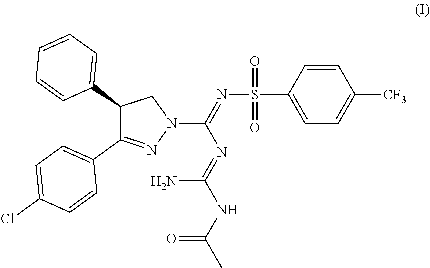 [SMILES: CC(=O)N/C(N)=N/C(=N\S(=O)(=O)c1ccc(C(F)(F)F)cc1)N1C[C@H](c2ccccc2)C(c2ccc(Cl)cc2)=N1]

to the human subject for a period of at least 28 days.
Embodiment 30. The method of any of the preceding embodiments, wherein the subject presents one or more of the following:
   i. fasting triglycerides >1.5 mmol/L for males and females and/or
   ii. an OGTT indicating impaired glucose tolerance as indicated by a 2-hour value >140 mg/dl or any value >200 mg/dl at any time point or a HbA1C level ≥5.7% but ≤6.4%.
Embodiment 31. The method of any of the preceding embodiments, wherein the subject exhibits no significant changes relative to baseline for bilirubin, AST and/or ALT after 28 days of treatment with the compound of formula I.
Embodiment 32. The method of any of the preceding embodiments, wherein the subject exhibits a body weight reduction relative to baseline between about 0.1 and about 7%, between about 1 and about 6%, between about 2 and about 5%, between about 3 and about 5%, about 4%, about 3.5%, about 3% or about 2.5% after 28 days of treatment with the compound of formula I.
Embodiment 33. The method of any of the preceding embodiments, wherein the subject exhibits a triglycerides reduction relative to baseline between about 1 and about 10%, between about 2 and about 9%, between about 3 and about 8%, about 8%, about 7%, about 6% or about 5% after 28 days of treatment with the compound of formula I.
Embodiment 34. The method of any of the preceding embodiments, wherein the subject exhibits a change in leptin levels relative to baseline of about between 10% and about 20%, after 28 days of treatment with the compound of formula I.
Embodiment 35. The method of any of the preceding embodiments, wherein the subject exhibits a reduction in LDL level relative to baseline of at least about 0.1 mmol/L to about 1 mmol/L after 28 days of treatment with the compound of formula I.
Embodiment 36. The method of any of the preceding embodiments, wherein the subject exhibits a reduction in VLDL level relative to baseline of at least about 0.03 mmol/L to about 1 mmol/L after 28 days of treatment with the compound of formula I.
Embodiment 37. The method of any of the preceding embodiments, wherein the subject exhibits a reduction in total cholesterol levels relative to baseline of at least about 0.1 mmol/L to about 1 mmol/L after 28 days of treatment with the compound of formula I.
Embodiment 38. The method of any of the preceding embodiments, wherein the compound of Formula I is for oral administration to the subject at a dose of 25 mg or more.
Embodiment 39. The method of any of the preceding embodiments, wherein the compound of Formula I is for daily oral administration to the subject at a dose of 25 mg or more.
Embodiment 40. The method of any of the preceding embodiments, wherein the compound of Formula I is for daily oral administration to the subject at a dose of about 25 mg.
Embodiment 41. The compound for use, use or method of any of the preceding embodiments wherein the compound of Formula I is for daily oral administration to the subject at a dose of less than 25 mg.
Embodiment 42. Use of a compound of Formula I or a pharmaceutically acceptable salt thereof:

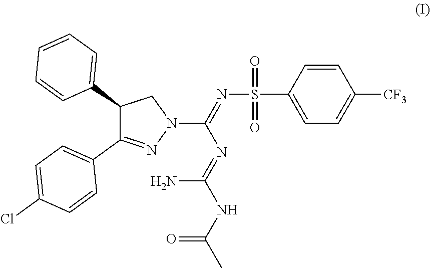 [SMILES: CC(=O)N/C(N)=N/C(=N\S(=O)(=O)c1ccc(C(F)(F)F)cc1)N1C[C@H](c2ccccc2)C(c2ccc(Cl)cc2)=N1]

to improve or reduce glucose intolerance in a subject, wherein the compound of Formula I, wherein said improving or reducing is relative to baseline level.
Embodiment 43. Use of a compound of Formula I or a pharmaceutically acceptable salt thereof:

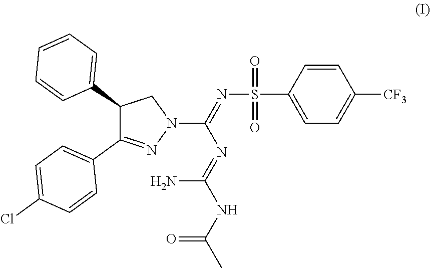 [SMILES: CC(=O)N/C(N)=N/C(=N\S(=O)(=O)c1ccc(C(F)(F)F)cc1)N1C[C@H](c2ccccc2)C(c2ccc(Cl)cc2)=N1]

to improve the plasma lipid profile relative to baseline level (e.g. total cholesterol, HDL, LDL, VLDL, and/or triglycerides) in a subject, wherein said improving is relative to baseline level.
Embodiment 44. Use of a compound of Formula I or a pharmaceutically acceptable salt thereof:

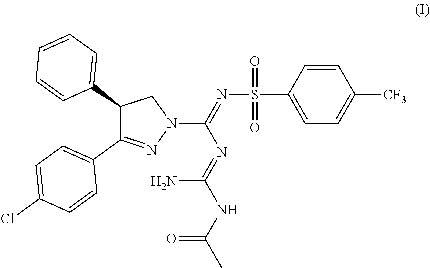 [SMILES: CC(=O)N/C(N)=N/C(=N\S(=O)(=O)c1ccc(C(F)(F)F)cc1)N1C[C@H](c2ccccc2)C(c2ccc(Cl)cc2)=N1]

to reduce LDL, VLDL, and/or triglycerides in a subject, wherein said reduction is relative to a baseline level.
Embodiment 45. Use of a compound of Formula I or a pharmaceutically acceptable salt thereof:

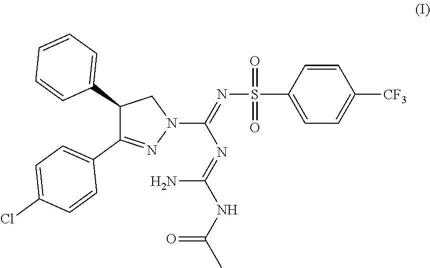 [SMILES: CC(=O)N/C(N)=N/C(=N\S(=O)(=O)c1ccc(C(F)(F)F)cc1)N1C[C@H](c2ccccc2)C(c2ccc(Cl)cc2)=N1]

to reduce subjective appetite sensations in a subject having a metabolic syndrome, wherein said reduction is relative to a baseline level.
Embodiment 46. The use of embodiment 45, wherein the reduction of subjective appetite sensations is determined by an Appetite and Food Intake Questionnaire.
Embodiment 47. The use of any of the preceding embodiments 42-46, wherein the subject is a human subject and presents one or more of the following:
waist circumference ≥88 cm for female subjects or ≥102 cm for male subjects;
fasting triglyceride (e.g. >1.5 mmol/L for males and females) and/or
impaired glucose tolerance (e.g. an OGTT indicating impaired glucose tolerance as indicated by a 2-hour value >140 mg/dl or any value >200 mg/dl at any time point or a HbA1C level ≥5.7% but ≤6.4%.)
Embodiment 48. The use of any of the preceding embodiments 42-47, wherein the compound of Formula I is for oral administration to the subject.
Embodiment 49. The use of any of the preceding embodiments, wherein the compound of Formula I is for oral administration to the subject at a dose of 25 mg or more.
Embodiment 50. The use of any of the preceding embodiments 42-48, wherein the compound of Formula I is for daily oral administration to the subject at a dose of 25 mg or more.
Embodiment 51. The use of any of the preceding embodiments 42-49, wherein the compound of Formula I is for daily oral administration to the subject at a dose of about 25 mg.
Embodiment 52. A method for improving or reducing glucose intolerance in a glucose intolerant human subject, said method comprising administering a compound of Formula I or a pharmaceutically acceptable salt thereof:

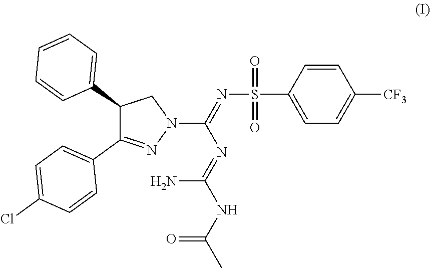 [SMILES: CC(=O)N/C(N)=N/C(=N\S(=O)(=O)c1ccc(C(F)(F)F)cc1)N1C[C@H](c2ccccc2)C(c2ccc(Cl)cc2)=N1]

to a subject in need thereof, wherein said improving or reducing is relative to baseline level.
Embodiment 53. A method for improving the plasma lipid profile (e.g. total cholesterol, HDL, LDL, VLDL, and/or triglycerides) in a subject in need thereof, said method comprising administering a compound of Formula I or a pharmaceutically acceptable salt thereof:

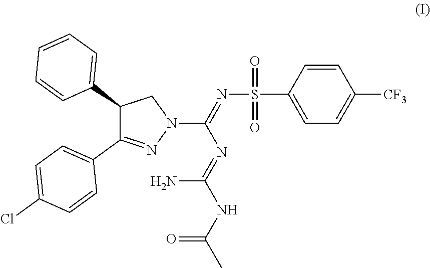 [SMILES: CC(=O)N/C(N)=N/C(=N\S(=O)(=O)c1ccc(C(F)(F)F)cc1)N1C[C@H](c2ccccc2)C(c2ccc(Cl)cc2)=N1]

to the subject, wherein said improving is relative to baseline level.
Embodiment 54. A method for reducing LDL, VLDL, and/or triglycerides in a subject in need thereof, said method comprising orally administering a compound of Formula I or a pharmaceutically acceptable salt thereof:

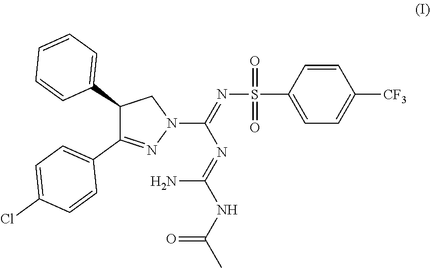 [SMILES: CC(=O)N/C(N)=N/C(=N\S(=O)(=O)c1ccc(C(F)(F)F)cc1)N1C[C@H](c2ccccc2)C(c2ccc(Cl)cc2)=N1]

to the subject, wherein said reducing is relative to baseline level.
Embodiment 55. A method for reducing subjective appetite sensations in a subject in a subject in need thereof, said method comprising administering a compound of Formula I or a pharmaceutically acceptable salt thereof:

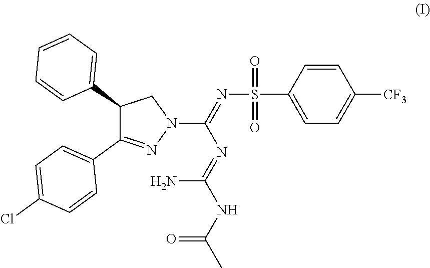 [SMILES: CC(=O)N/C(N)=N/C(=N\S(=O)(=O)c1ccc(C(F)(F)F)cc1)N1C[C@H](c2ccccc2)C(c2ccc(Cl)cc2)=N1]

to the subject, wherein said reducing is relative to baseline level.
Embodiment 56. The method of embodiment 55, wherein the reduction of subjective appetite sensations is determined by an Appetite and Food Intake Questionnaire.
Embodiment 57. The method of any of the preceding embodiments 52-56, wherein the subject is a human subjects and presents one or more of the following:
waist circumference ≥88 cm for female subjects or ≥102 cm for male subjects;
fasting triglyceride (e.g. >1.5 mmol/L for males and females) and/or
impaired glucose tolerance (e.g. an OGTT indicating impaired glucose tolerance as indicated by a 2-hour value >140 mg/dl or any value >200 mg/dl at any time point or a HbA1C level ≥5.7% but ≤6.4%.)
Embodiment 58. The method of any of the preceding embodiments 52-57, wherein the compound of Formula I is for oral administration to the subject.
Embodiment 59. The method of any of the preceding embodiments, wherein the compound of Formula I is for oral administration to the subject at a dose of 25 mg or more.
Embodiment 60. The method of any of the preceding embodiments 52-58, wherein the compound of Formula I is for daily oral administration to the subject at a dose of 25 mg or more.
Embodiment 61. The method of any of the preceding embodiments 58-59, wherein the compound of Formula I is for daily oral administration to the subject at a dose of about 25 mg.

### Experiments and Examples

A phase 1b study to examine the pharmacokinetic and pharmacodynamic effects of INV-202 in subjects with metabolic syndrome as defined by hypertriglyceridemia, abdominal obesity, and impaired glucose tolerance over 28 days was performed.

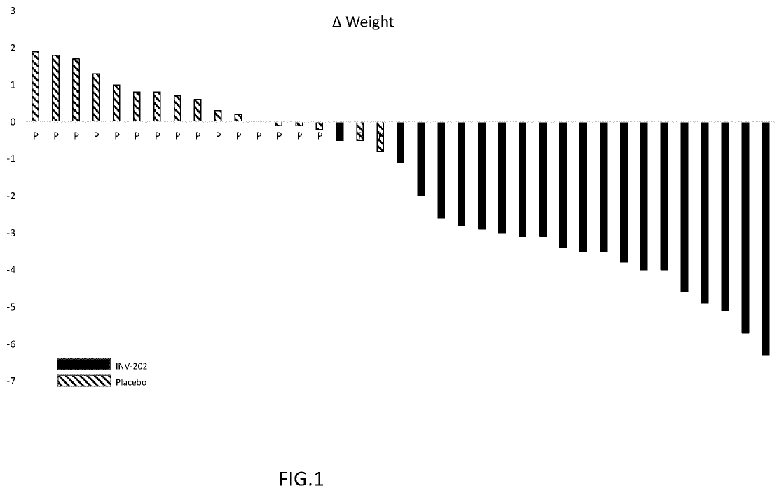

Results are reported in Tables 1-5 and FIG. 1.

### Objectives

To examine the PK of INV-202 in subjects with evidence of metabolic syndrome over 28 days.
To examine the PD effects of INV-202 as measured by glucose metabolism and lipid profile over 28 days.
To evaluate the safety and tolerability of INV-202 over 28 days.

### Endpoints

### PK Endpoints:

The PK parameters of INV-202 and its major metabolite(s).

Details are available in the PK Section of the protocol and additional details will be included in the SAP.

### PD Endpoints:

0-3 hour AUC blood glucose level during the OGTT
Change from baseline in:
   Plasma insulin level
   C-peptide level
   Plasma lipid profile (HDL, LDL, VLDL, and triglycerides)
   Adiponectin and leptin
   hs-CRP
   Weight
   Waist circumference
   Appetite and Food Intake questionnaire score
   Urine albumin to creatinine ratio

### Safety Endpoints:

AEs: Frequency, severity, time to onset, duration, and relatedness to study drug
SAEs: Frequency, severity, time to onset, duration, and relatedness to study drug
Discontinuations due to AEs
Changes in clinical laboratory parameters

### Study Design

This is single center, Phase 1B, randomized, double-blind, placebo-controlled, 28-day repeat dose study.

The study includes subjects with evidence of metabolic syndrome and glucose intolerance that are randomized 1:1 to receive either INV-202 or placebo. 20 subjects will receive 25 mg of INV-202 and 20 subjects will receive a matching placebo daily for 28 consecutive days, for a total of 40 subjects.

### Study Population

### Rationale for the Study Population

This study will enroll subjects with metabolic syndrome as defined by hypertriglyceridemia, abdominal obesity, and impaired glucose tolerance.

The effect of INV-202 on body weight and food consumption was studied in a DIO mouse model. Following 10 days of dosing, INV-202 was shown to dose dependently reduce body weight. INV-202 also exhibited decreased glucose AUC in an OGTT test, showed significant effects in reducing C-peptide levels and insulin levels, and also significantly decreased ALT and liver triglyceride following 28-days of repeat dosing.

No reproductive studies in animals have been conducted with INV-202 so far. Since the risk to the developing human foetus following exposure to INV-202 is unknown at this time, only males and non-pregnant, non-lactating females will be included in the study. As a precaution for female subjects, pregnancy tests will be performed at different times during the study. Females of childbearing potential will be included if they use appropriate methods of contraception.

In addition, non-surgically-sterile male subjects who are sexually active with non-sterile female partners will be required to use effective contraceptive methods for the duration of the study and for 90 days following the last administration of the study drug. All male subjects (including men who have had vasectomies) with a pregnant partner must agree to use a condom from (the first, if applicable) dosing until at least 90 days after (the last, if applicable) study drug administration. In addition, all male subjects must be willing not to donate sperm until 90 days following (the last, if applicable) study drug administration.

### Sample Size

It is planned to enroll approximately 40 male or female subjects for participation in this study.

The study will consist of one cohort, randomized 1:1 to receive either INV-202 or placebo. 20 subjects will receive INV-202 and 20 subjects will receive a matching placebo daily for 28 consecutive days, for a total of 40 subjects.

The sample size of this study is not determined based on statistical calculations, it is rather determined based on the probability of observing an AE. This number of subjects is judged adequate to achieve the study objectives.

### Inclusion Criteria

Subjects enrolled in this study will be members of the community at large. Subjects must meet all of the following criteria to be included in the study:
1) Provision of a signed and dated ICF.
2) Willing and able to comply with all study procedures for the duration of the study.
3) Male or female, ≥18 and ≤65 years of age.
4) Waist circumference ≥88 cm for female subjects or ≥102 cm for male subjects.
5) Fasting triglyceride >1.5 mmol/L for males and females.
6) An OGTT indicating impaired glucose tolerance as indicated by a 2-hour value >140 mg/dl or any value >200 mg/dl at any time point or a HbA1C level ≥5.7% but ≤6.4%.
7) Females of childbearing potential who are sexually active with a non-sterile male partner (sterile male partners are defined as men vasectomized at least 6 months prior to the first study drug administration) must be willing to use one of the following acceptable contraceptive methods throughout the study and for at least 30 days after the last study drug administration:
   a) Simultaneous use of hormonal contraceptives, started at least 4 weeks prior to study drug administration and must agree to use the same hormonal contraceptive throughout the study, and condom for the male partner;
   b) Simultaneous use of intra-uterine contraceptive device, placed at least 4 weeks prior to study drug administration, and condom for the male partner;
   c) Simultaneous use of diaphragm or cervical cap with intravaginally applied spermicide and male condom for the male partner, started at least 21 days prior to study drug administration.
8) Females of non-childbearing potential must be:
   a) Post-menopausal (absence of menses for at least 12 months prior to the first study drug administration) with confirmation of the post-menopausal status by documented FSH level ≥40 mIU/mL; or
   b) Surgically sterile (complete hysterectomy, bilateral oophorectomy or tubal ligation at least 6 months prior to the first study drug administration).
9) Male subjects who are not vasectomized for at least 6 months, and who are sexually active with a female partner of childbearing potential (childbearing potential females are defined as women that are neither post-menopausal nor surgically sterile) must be willing to use one of the following acceptable contraceptive methods from the first study drug administration until at least 90 days after the last study drug administration:
   a) Simultaneous use of a male condom and, for the female partner, hormonal contraceptives used since at least 4 weeks or intra-uterine contraceptive device placed since at least 4 weeks;
   b) Simultaneous use of a male condom and, for the female partner, a diaphragm or cervical cap with intravaginally applied spermicide.
10) Male subjects (including men who have had a vasectomy) with a pregnant partner must agree to use a condom from the first study drug administration and for 90 days after the last study drug administration.
11) Male subjects must be willing not to donate sperm for 90 days following the last study drug administration.

### Exclusion Criteria

Subjects to whom any of the following applies will be excluded from the study:
1) Female who is lactating at screening.
2) Female who is pregnant according to a pregnancy test at screening or prior to study drug administration.
3) History of significant hypersensitivity to the study drug or excipients of the study drug.
4) History of severe hypersensitivity reactions, such as anaphylaxis.
5) History of rare hereditary problems of galactose and/or lactose intolerance, lactase deficiency or glucose-galactose malabsorption.
6) Positive screening results to HIV antigen and antibody, HBsAg or HCV tests.
7) Poses a significant suicidal risk as defined by C-SSRS score >Type 1 ideation at screening.
8) Any clinically significant illness in the 28 days prior to study drug administration.
9) Presence or history of significant gastrointestinal, liver or kidney disease, or surgery that may affect drug bioavailability.
10) History of significant and uncontrolled or unstable cardiovascular, pulmonary, hematologic, neurological, psychiatric, endocrine, immunologic or dermatologic disease.
11) History of seizures (epilepsy) of any kind.
12) History of cranial surgery.
13) Presence of clinically significant ECG abnormalities at the screening visit, as defined by medical judgment (maximum QTcF 450 msec).
14) Any other clinically significant abnormalities in laboratory test results at screening that would, in the opinion of an investigator, increase the subject's risk of participation, jeopardize complete participation in the study, or compromise interpretation of study data. Mild elevations in AST/ALT as may be seen in NAFLD are not exclusionary. However, in these subjects, please follow the hepatic safety section.
15) New prescription medication or changes to medication regimen within 90 days prior to the first dose. (i.e., stable doses of antihypertensives etc. are allowed).
16) Use of the following medications for the timeframes specified below, with the exception of medications exempted by the Investigator on a case-by-case basis because they are judged unlikely to affect the PK profile of the study drug or subject safety (e.g., topical drug products without significant systemic absorption):
   a) Any vaccination, including COVID-19 vaccine, within 14 days prior to the first dose;
   b) Depot injection or implant of any drug within 3 months prior to the first dose;
   c) Any drugs known to induce or inhibit hepatic drug metabolism within 30 days prior to the first dose.
   d) Any drugs for the treatment of diabetes within 30 days prior to the first dose.
   e) Any drugs prohibited by the Investigator on a case-by-case basis because they are judged likely to affect the PD profile of the study drug or subject safety, within at least 5 times the half-life of the drug and a minimum of 30 days prior to the first dose.
17) Positive urine drug screen or alcohol breath test at screening.
18) History of significant alcohol abuse within 1 year prior to screening or regular use of alcohol within 6 months prior to the screening visit (more than 14 units of alcohol per week [1 unit=150 mL of wine, 360 mL of beer, or 45 mL of 40% alcohol]).
19) History of significant drug abuse within 1 year prior to screening, use of soft drugs within 3 months prior to screening, marijuana within 1 month prior to screening, or hard drugs (such as cocaine, PCP, crack, opioid derivatives including heroin, and amphetamine derivatives) within 1 year prior to screening.
20) Use of any cannabinoid containing product, including cannabis, within 1 month prior to screening, by any route (e.g., oral, inhaled, topical).
21) Participation in a clinical research study involving the administration of an investigational or marketed drug or device within 30 days (or a minimum of 5 half-lives, whichever is longer) prior to the first dose, administration of a biological product in the context of a clinical research study within 90 days prior to the first dose, or concomitant participation in an investigational study involving no drug or device administration.
22) Donation of plasma within 7 days prior to dosing. Donation or loss of blood (excluding volume drawn at screening) of 50 mL to 499 mL of blood within 30 days, or more than 499 mL within 56 days prior to the first dose.
23) Any reason, which in the opinion of the Investigator, would prevent the subject from participating in the study.

### Clinical Procedures

Unless otherwise specified, procedures, data collection and evaluation will be conducted as per the clinical site SOPs.

### Screening Procedures

Subject screening procedures will be performed within 35 days preceding administration of study medication. Subjects must provide written informed consent prior to initiation of any screening procedures. The consent to perform some general screening procedures may be obtained on a consent document other than the ICF specific to this study, and therefore, some screening test results could be obtained before signature of the ICF specific to this study. The study-specific ICF must be signed and dated by the subject before participation in study-specific procedures.

Screening procedures will include: Demographic data, medical and medication histories, complete physical examination, body measurements, C-SSRS, OGTT, vital signs (BP, HR, RR, and OT), 12-lead ECG, hematology, biochemistry, coagulation, endocrinology (only FSH), serology (HIV antigen and antibody, HBsAg, and HCV antibody), urinalysis, serum pregnancy test, alcohol breath test, and urine drug screen.

FSH levels will be measured at screening, unless documented results are available within 6 months preceding dosing. For women using hormone replacement therapy and for whom FSH levels have to be confirmed, these tests will be performed at least one week following the withdrawal of the therapy, but before the first dosing.

Subjects must meet all inclusion and exclusion criteria and have evidence of glucose intolerance. The OGTT will not need to be performed at screening if there is evidence of HbA1C≥5.7% but ≤6.4% obtained within the last 3 months. If there is no such evidence, subjects will undergo an OGTT consisting of a 75 g glucose load with blood draws at 30 minutes, 1 hour, and 2 hours post-intake.

For eligibility purposes, abnormal laboratory or vital signs results may be repeated once if an abnormal result is observed at the initial reading. Moreover, abnormalities found in the ECG may need to be confirmed by repeated measurements. In the event that the participation of a subject in the study is delayed and some screening procedures had been performed outside of the prescribed screening window, outdated screening procedures can be repeated.

### Study Visits

The participation of subjects in this study should last approximately 5-6 weeks. Subjects will undergo a screening visit and 7 study visits:

### First Baseline Visit on Day −1:

Subjects will arrive at the clinic following at least an 8-hour overnight fast.

Subjects will undergo an OGTT consisting of a 75 g glucose load with blood draws pre-load and at 30 minutes, 1 hour, 2 hours, and 3 hours post-intake.

If the screening C-SSRS was performed more than 21 days prior to Day −1, a repeat C-SSRS should be done on Day −1. Otherwise, the screening C-SSRS may be used as the baseline assessment.

### Second Baseline Visit on Day 1:

Subjects will arrive at the clinic following at least an 8-hour overnight fast.

Blood and urine will be collected pre-dose for baseline lab work and PK/PD assessments. The subjects' weight and waist circumference will be measured, and they will undergo additional assessments as outlined in section 0 including: a brief physical examination, vital sign measurement, and 12-lead ECG.

Subjects will fill out the Appetite and Food Intake questionnaire.

At this visit, subjects will be randomized at a ratio of 1:1 to receive either INV-202 25 mg or matching placebo. Doses of INV-202 (or matching placebo) sufficient for one week will be dispensed to the subjects. Food will be provided, and the first dose will be taken at the site with food. Subjects will be instructed to self-administer doses of the study drug once daily with food and record the time of dosing, any observations regarding the taste of the tablets, as well as any AEs that they may experience, in a subject diary card. The subjects will then be discharged from the site.

### Weekly Study Visits on Days 8, 15, and 22:

Once weekly, approximately every seven days, subjects will return to the clinic following at least an 8-hour overnight fast for PK/PD, safety, and tolerability assessments.

Blood and urine will be collected pre-dose for lab work and PK/PD assessments. The subjects' weight and waist circumference will be measured, preferably using the same instruments as the baseline measurement. They will undergo additional assessments as outlined in section 0 including: a brief physical examination and vital signs. Diary cards will be collected and reviewed with the subjects to document AEs, comments, and time of drug administration.

Subjects will fill out the Appetite and Food Intake questionnaire.

Doses of INV-202 (or matching placebo) sufficient for one week will be dispensed to the subjects. Food will be provided, and the daily dose will be taken at the site with food. Subjects will be instructed to self-administer doses of the study drug once daily with food and record the time of dosing, any observations regarding the taste of the tablets, as well as any AEs that they may experience, in a subject diary card. The subjects will then be discharged from the site.

### EOT Visit on Day 29 (+2 Days):

Subjects will arrive at the clinic following at least an 8-hour overnight fast for PK/PD, safety, and tolerability assessments.

Blood and urine will be collected for lab work and PK/PD assessments. The subjects' weight and waist circumference will be measured, preferably using the same instruments as the baseline measurement. They will undergo additional assessments as outlined in section 0 including: a brief physical examination, vital sign measurement, and 12-lead ECG. Diary cards will be collected and reviewed with the subjects to document AEs, comments, and time of drug administration.

Subjects will undergo an OGTT consisting of a 75 g glucose load with blood draws at 30 minutes, 1 hour, 2 hours, and 3 hours post-intake. Subjects will also fill out the Appetite and Food Intake questionnaire, and the C-SSRS questionnaire will be administered. The subjects will then be discharged from the site.

### EOS Visit on Day 35 (+2 Days):

Approximately 6 days following the Day 29 (+2 days) visit, subjects will arrive at the clinic following at least an 8-hour overnight fast for a final blood draw and urine collection for lab work and PK/PD assessments. Additional assessments will be preformed as outlined in section 0 including: a complete physical examination, vital sign measurement, and 12-lead ECG. Subjects will fill out the C-SSRS questionnaire.

At the end of this visit, the subjects will have completed their participation in the study and can then be discharged from the site.

### Randomization and Blinding

Subjects eligible for participation will be randomized (on Day 1) to receive either the active study drug or matching placebo in a 1:1 ratio, for a total of 20 subjects receiving INV-202 and 20 subjects receiving the matching placebo. One randomization scheme will be produced for the study.

The subjects and the clinical personnel involved in the collection, monitoring, revision, or evaluation of AEs, or personnel who could have an impact on the outcome of the study will be blinded with respect to the subject's treatment assignment (INV-202 or placebo). Blinding will be maintained at least until the clinical phase of the study is completed (i.e., when reporting and evaluation of all AEs have been completed).

Designated pharmacy personnel at the clinical site not directly involved with the clinical aspects of the trial will prepare and dispense the study medication and will be aware of the randomization code. The study drug and placebo will have the same visual appearance in order to avoid compromising the study blinding.

In the event of an emergency for an individual subject in which knowledge of the study treatment is critical to the subject's medical management, the Investigator may break the blind for that subject. An envelope for each subject containing his/her treatment assignment will be available from the pharmacy personnel. The Investigator or other attending study physician will make every effort to contact the Sponsor prior to unblinding a subject's treatment assignment and will record the date and reason for unblinding in the study source documents.

The pharmacy personnel will provide to the bioanalytical laboratory a list of subject numbers to be analysed for each dose level, so that only samples from subjects who received the active drug are analysed.

### Study Medication

Active Study Drug: INV-202, 25 mg tablets (manufactured by Inversago Pharma Inc., Canada), given as 1×25 mg tablet administered orally.

Placebo: Tablets identical in appearance color, shape and size to the active INV-202 (manufactured by Inversago Pharma Inc., Canada), administered orally.

### Drug Supplies and Accountability

It is the responsibility of the Sponsor to ensure that study medications provided for this study are manufactured under Good Manufacturing Practice (GMP) and are suitable for human use. The Sponsor is responsible to ship a sufficient amount of dosage units to allow the clinical site to maintain an appropriate sampling for the study.

Study medication will be stored at the clinical site as per applicable requirements. The medications will be stored in a locked, temperature-controlled medication room with restricted access. Container(s) will bear a label containing at least the name of the study drug, lot and/or batch number, and manufacturing and/or expiry/retest date.

Individual doses for each subject will be dispensed at the clinical site, as per appropriate SOP. Individual doses will be dispensed according to the randomization scheme in appropriate envelopes/containers indicated with at least the project number and the subject number/spare number.

All study drug received at the site will be inventoried and accounted for throughout the study and the result recorded in the drug accountability/retention record according to the clinical site appropriate SOP.

### Study Drug Administration

Subjects will receive a daily oral dose of INV-202 25 mg or a matching placebo for 28 consecutive days.

At the baseline visit and follow-up visits on Days 8, 15, and 22, no food will be allowed from at least 8 hours prior to arrival to the clinic until the completion of all scheduled assessments.

After completion of the assessments, food will be provided, and the daily dose will be taken at the site with food. The study medication will be administered to each subject with 240 mL of water and a hand and mouth check will be performed to ensure consumption of the medication. Time of dosing will be set equal to the time when the tablet is administered to the subject.

For all other doses, subjects will take the study drug independently and will be instructed to self-administer doses of the study drug once daily with food. Subjects will be required to record the time of dosing and any observations regarding the taste of the tablets in a subject diary card.

Each study drug administration will be separated by approximately 24 hours and should be done at approximately the same time every day. If a study drug dose has been missed, and the subject becomes aware of this prior to 8 μm, the missed dose may still be taken. If the subject becomes aware of the missed dose after 8 pm, that dose should be skipped, and the next dose should be taken at the regular time.

When subjects return to the clinic on Days 8, 15, 22 and 29, they will be required to bring with them any unused study drug and/or empty study drug containers. A post-dose inventory will be performed on the dose containers.

### Study Restrictions

### Food and Fluids

The subjects will be required to abstain from:
Food or beverages containing grapefruit, starfruit, pomegranate, pineapple, or pomelo from 7 days pre-dose until after the last PK blood sample collection of the study;

At all study visits, no food will be allowed from at least 8 hours prior to arrival to the clinic until the completion of all scheduled assessments. Food will be provided after completion of the assessments. Water will be provided ad libitum at all times.

### Alcohol, and Illicit Drugs

Subjects will be required to abstain from using soft or hard drugs from screening and throughout the study.

Consumption of alcohol-based products will be prohibited from 8 hours prior to each visit. Subjects will be asked to limit alcohol intake to a moderate level (<2 drinks per day) from Day −1 until study completion.

### Concomitant Medications

Subjects will be required to avoid new prescription medication or changes to medication regimens, as well as drugs likely to alter the PD profile of the study drug (e.g., diabetes medication, etc.) as outlined in exclusion criteria 15)16) and throughout the study.

No new concomitant medications or changes to medication regimens are allowed from 90 days prior to the first dose until after the last PK/PD blood sample collection of the study, with the exception of one(s) required for the medical management of an AE, medications exempted by the Investigator on a case-by-case basis that are judged unlikely to affect the PK profile of the study drug or subject safety (e.g., topical drug products without significant systemic absorption) and occasional use of acetaminophen.

Subjects will also be required to avoid receiving any vaccination, including COVID-19 vaccine, from 14 days prior to dosing and throughout the study. If vaccination is required for any reason, it must first be discussed with and exempted by the Investigator on a case-by-case basis to ensure that it does not compromise the PK profile of the study drug or the subject safety.

All medications taken by subjects after screening until the last study day will be documented as concomitant medications. Any concomitant medication use, other than the allowed medications stated above, will be reviewed and evaluated on a case-by-case basis by the Investigator to determine whether they have potential impact on a subject's eligibility, continued participation in the study, or the study results.

### Posture and Physical Activity

An OGTT will be performed at screening, on Day −1, and on Day 29 (+2 days). Since activity can interfere with test results, subjects will be required to remain seated, unless medically necessary or required by procedures, from the time the glucose load is administered until the last blood draw (2-hours post-intake at screening and 3 hours post-intake at other visits) is completed.

### Subject Monitoring

Subjects will be monitored throughout the study by the clinical staff for AEs. The Investigator or designee will be on site for the first study drug administration. If necessary, the Investigator or designee at the clinical site or a healthcare professional in a nearby hospital will administer treatment for any AE(s). A crash cart or emergency bag containing the necessary rescue material and appropriate medications will be available in the clinic to allow rapid intervention in case of emergency.

Safety parameters, including laboratory results and ECG, will be assessed by the Investigator or designee, using the clinical site's criteria for biomedical laboratory and ECG acceptance ranges as suggested guidelines in making the medical assessment.

Scheduled safety measurements will be repeated according to the clinical site SOPs or upon request from the Investigator or designee. Any abnormal repeated measurement will be evaluated by the Investigator or designee and repeated if judged necessary. Further action may be taken upon the Investigator or designee's request.

Unless otherwise specified or for subject safety, when the timing of multiple procedures coincides, the following priority order should be adhered to, whenever possible: vital signs, ECGs, blood samples for PK/PD analysis, clinical laboratory samples, physical examination, OGTT, Appetite and Food Intake questionnaire, C-SSRS questionnaire and diary card review.

Subjects will be advised to notify their healthcare professional(s) (e.g., physician, dentist, and/or pharmacist) that they are participating in a clinical research study on a drug called INV-202 before taking any medicines or undergoing any medical procedure.

### Physical Examination

A complete physical examination will be performed at screening and at the FU Visit/EOS/ET on Day 35 (+2 days). A brief physical examination will be performed pre-dose on Days 1, 8, 15, and 22, and on Day 29 (+2 days). Other physical examinations will be carried out at the discretion of the Investigator.

The complete physical examination will include assessments of the following: head, eyes, ears, nose, throat (HEENT), lymph nodes, neck, chest, lungs, abdomen, musculoskeletal, dermatological, cardiovascular/peripheral vascular, and general neurological examination. The brief physical examination will include assessments of the following: HEENT, chest, lungs, abdomen, dermatological, cardiovascular/peripheral vascular, and areas of note elicited from the subject.

### Body Measurements

Body measurements performed at screening will include height and weight measurements, waist circumference, as well as BMI calculation. Height will only be measured at screening. Weight and waist circumference will also be measured pre-dose on Days 1, 8, 15, and 22, and on Day 29 (+2 days). BMI will be calculated as needed.

Weight and waist circumference will be measured in triplicate using a standard measuring method. The same scale should be used at all visits for a given subject. The scale should be calibrated according to the manufacturer's recommendation. A medical grade scale for weight should be used.

Waist circumference should be measured at the level of the umbilicus and around the top of the iliac crest. The same measuring device should be used at all visits for a given subject. The tape should go around the subject and should be parallel to the floor in the plane that includes the umbilicus and the top of the iliac crest.

### Vital Signs

BP, HR, RR and OT will be measured at screening and pre-dose on Days 1, 8, 15, and 22, on Day 29 (+2 days), and at the FU Visit/EOS/ET on Day 35 (+2 days). Vital signs will be measured in a sitting position (except for safety reasons and when ECG are planned around the same time). When vital signs measurements coincide with a blood draw, they should preferably be performed before the blood collection, whenever possible.

### Safety ECG

A 12-lead ECG will be performed at screening, pre-dose on Day 1, on Day 29 (+2 days), and at the FU Visit/EOS/ET on Day 35 (+2 days). Standard 12-lead ECGs will be performed after subjects have been supine for at least 5 minutes. When ECG coincides with a blood draw, it should preferably be performed before the blood collection, whenever possible.

### Drug and Alcohol Screen

A urine drug screen (amphetamines, methamphetamines, barbiturates, benzodiazepines, tetrahydrocannabinol, cocaine, opiates, PCP, 3,4-methylenedioxymethamphetamine [MDMA], methadone), and an alcohol breath test will be performed at screening.

### Pregnancy Test

A serum pregnancy test will be performed at screening. A urine pregnancy test will be performed pre-dose on Day 1, and at the FU Visit/EOS/ET on Day 35 (+2 days).

A urine pregnancy test that yields a positive result will be confirmed by a serum pregnancy test. Dosing will be halted if a urine pregnancy test is positive. If a serum pregnancy test confirms the result, dosing will be discontinued, otherwise, dosing may proceed.

### Laboratory Assessments

Laboratory assessments must be performed following at least an 8-hour fast.

### Hematology

Hematology will be performed at screening, pre-dose on Days 1, 8, 15, and 22, on Day 29 (+2 days), and at the FU Visit/EOS/ET on Day 35 (+2 days).

The following will be assessed: complete blood count with differential, hemoglobin, and hematocrit, mean corpuscular volume, red blood cell morphology, white blood cell morphology, platelet count, platelet morphology.

### Biochemistry

Biochemistry will be performed at screening, pre-dose on Days 1, 8, 15, and 22, on Day 29

(+2 days), and at the FU Visit/EOS/ET on Day 35 (+2 days).

The following will be assessed: sodium, potassium, chloride, glucose, blood urea nitrogen, creatinine, creatinine clearance, direct bilirubin, indirect bilirubin, bilirubin total, ALP, AST, ALT, albumin, lactate dehydrogenase, calcium, lipase, amylase, phosphorus, HbA1C, serum ferritin and total protein.

In addition, a lipid profile will also be assessed: total cholesterol, HDL, LDL, VLDL and triglycerides.

Estimated glomerular filtration rate (eGFR), expressed in mL/min/1.73 m² of body surface area, will be calculated using the Modification of Diet in Renal Disease (MDRD4: 4-variable) equation.

Considering that indirect bilirubin is calculated from total and direct bilirubin values, indirect bilirubin result would not be available in case direct bilirubin is below the limit of quantification.

### Coagulation

Coagulation will be performed at screening, pre-dose on Days 1, 8, 15, and 22, on Day 29 (+2 days), and at the FU Visit/EOS/ET on Day 35 (+2 days).

The following will be assessed: INR, activated partial thromboplastin time (aPTT), prothrombin time (PT), partial thromboplastin time (PTT), and partial thromboplastin time control.

### Endocrinology

Endocrinology will be performed at screening, pre-dose on Days 1, 8, 15, and 22, on Day 29

(+2 days), and at the FU Visit/EOS/ET on Day 35 (+2 days).

The following will be assessed only at screening: FSH level will be measured for post-menopausal females, and the post-menopausal status will be confirmed by documented FSH level ≥40 mIU/mL.

The following will be assessed only pre-dose on Days 1, 8, 15, and 22, on Day 29 (+2 days), and at the FU Visit/EOS/ET on Day 35 (+2 days): C-peptide, insulin, TSH, and T4.

### Serology

Serology will be performed at screening. The following will be assessed: HIV antigen and antibody, HBsAg, HCV antibody.

### Urinalysis

Urinalysis will be performed at screening, pre-dose on Days 1, 8, 15, and 22, on Day 29 (+2 days), and at the FU Visit/EOS/ET on Day 35 (+2 days).

The following will be assessed: macroscopic examination, pH, specific gravity, protein, glucose, ketones, bilirubin, occult blood, nitrite, urobilinogen, leukocytes, albumin, and creatinine (to determine the urine albumin to creatinine ratio). Microscopic examination will be performed as per laboratory standards according to internal procedures.

### Oral Glucose Tolerance Test

The OGTT will be performed at screening, on Day −1, and on Day 29 (+2 days). The OGTT will not need to be performed at screening if there is evidence of HbA1C≥5.7% but ≤6.4% obtained within the last 3 months.

For the OGTT performed at screening, a 75 g glucose load will be administered with blood draws at 30 minutes, 1 hour, and 2 hours.

For the OGTT performed on Day −1, a 75 g glucose load will be administered with blood draws pre-load, at 30 minutes, 1 hour, 2 hours and 3 hours post-intake.

For the OGTT performed on Day 29 (+2 days), a 75 g glucose load will be administered with blood draws at 30 minutes, 1 hour, 2 hours and 3 hours post-intake.

Since activity can interfere with test results, subjects will be required to remain seated, unless medically necessary or required by procedures, until the last blood draw is completed.

### Diary Card Review

Throughout the treatment period, subjects will record the time of dosing, any observations regarding the taste of the tablets, as well as any AEs that they may experience, in a subject diary card. Diary cards will be collected and reviewed on Days 8, 15, 22 and 29.

### Appetite and Food Intake Questionnaire

An Appetite and Food Intake questionnaire will be completed pre-dose on Days 1, 8, 15, and 22, and on Day 29 (+2 days). The questionnaire to be completed is presented in section Erreur!Source du renvoi introuvable.

### Columbia Suicidality Severity Rating Scale (C-SSRS)

A suicide risk assessment will be performed using the C-SSRS questionnaire at screening, Day −1 (if more than 21 days have passed since the screening C-SSRS), Day 29 (+2 days) and at the FU Visit/EOS/ET on Day 35 (+2 days).

This scale will be administered by a member of the medical team, completed on site, and will be on paper. The “Lifetime” C-SSRS questionnaire template will be used at the screening visit and on Day −1 (if applicable), and the “since last visit” C-SSRS questionnaire template will be used on Day 29 (+2 days) and at the FU Visit/EOS/ET. If the Investigator determines that a subject is at risk of suicide or self-harm, he/she must immediately be discontinued from the study and appropriate measures to ensure the subject's safety and obtain mental health evaluation must be implemented. The event should be recorded as either an AE or a SAE, as determined by the Investigator, and reported within 24 hours to the Sponsor.

### FU/EOS/ET Visit Procedures

A FU visit will occur on Day 35 (+2 days), approximately 168 hours following the last study drug administration.

EOS procedures are scheduled to be performed at the FU visit and will consists of the following assessments: complete physical examination, C-SSRS, vital signs, 12-lead ECG, hematology, biochemistry, coagulation, endocrinology, urinalysis, urine pregnancy test, PK/PD blood sampling and AE monitoring.

These procedures will be performed within 14 days after the last participation of the subject in the study in case of ET. Subjects who discontinue from the study prematurely will undergo an ET visit.

### Hepatic Safety Precautions

The following instructions are to be followed if liver function markers and clinical presentation suggest the onset of hepatic impairment during the study. The hepatic safety of all randomized subjects will be assessed through the use of the following algorithm based on FDA DILI management guidelines (Center for Drug Evaluation and Research (CDER), FDA. Guidance for Industry: Drug-Induced Liver Injury: Premarketing Clinical Evaluation. July 2009).

An increase of serum ALT or AST>3×ULN should be followed by repeat testing within 48 to 72 hours of all four of the usual serum measures (ALT, AST, ALP, and TBL) to confirm the abnormalities and to determine if they are increasing or decreasing. There also should be inquiry made about symptoms.

The need for prompt repeat testing is especially great if ALT or AST is much greater than 3×ULN and/or TBL is greater than 2×ULN.

If symptoms persist or repeat testing shows ALT or AST>3×ULN for subjects with normal baseline measures or 2-fold increases above baseline values for subjects with elevated values before drug exposure, it is appropriate to initiate close observation to determine whether the abnormalities are improving or worsening. Close observation includes repeat testing two or three times weekly, detailed medical and medication history, ruling out other causes, etc. If close monitoring is not possible, the drug should be discontinued. Moreover, INV-202 should be discontinued if:
ALT or AST>5×ULN
ALT or AST>3×ULN and (TBL>2×ULN or INR>1.5)
ALT or AST>3×ULN with the appearance of fatigue, nausea, vomiting, right upper quadrant pain or tenderness, fever, rash, and/or eosinophilia (>5%).

### Sample Collection and Processing

Plasma samples will be collected and processed as per the Analytical Methodology Information Sheet.

The total volume of blood including that collected for eligibility, safety purposes and repeat tests should not exceed approximately 250 mL.

### Blood Sample Collection for PK/PD Analysis

Blood samples for PK/PD analyses of INV-202 and its major metabolite(s), if applicable, will be collected as follows:

Approximately 6 blood samples will be collected for PK/PD assessments at the following time points: pre-dose on Days 1, 8, 15, and 22, on Day 29 (+2 days), and at the Day 35 (+2 days) FU/EOS/ET visit.

Intravenous cannulas may be used for blood collection to avoid multiple skin punctures, when appropriate. Otherwise, blood samples will be collected by direct venipuncture

### Data Collection and Evaluation

All clinical raw data will be recorded promptly, accurately, legibly, and indelibly by the clinical staff on raw data sheets and/or recorded electronically using validated software(s) and reported into Case Report Forms (CRFs). All raw data will be conserved in order to maintain data integrity. The Investigator and/or the clinical staff have the responsibility of ensuring the completeness and accuracy of the clinical data.

Details on the data management process will be described in a data management plan (DMP).

### Subject Withdrawal and Replacement

Subjects will be advised that they are free to withdraw from the study at any time. Over the course of the study, the Sponsor and the Investigator or designee may withdraw any subject from the study for one of the reasons described below; subject withdrawal will be done in accordance with the clinical site's SOP:
Safety reason;
Non-compliance with protocol requirements;
Significant protocol deviation;
Positive pregnancy test, drug screen, or alcohol test.

Clinical laboratory results will be reviewed by the Investigator or designee when available; subjects will be withdrawn from the study if it is deemed that the subject's safety may be at risk on the basis of these test results.

Subjects who withdraw or are withdrawn from the study after dosing will not be replaced.

However, in the event that the number of drop-outs exceeds initial expectations, subjects who withdraw or are withdrawn might be replaced at the discretion of the Sponsor. Such replacement resulting in dosing more subjects than planned in this protocol would be documented in a protocol amendment.

Subjects who withdraw or are withdrawn will be asked to remain at the clinic until the Investigator or designee agrees that the subject is fine and can be discharged. As soon as subject withdrawal is confirmed, blood sampling will be stopped. PK blood draws may be collected at the time of withdrawal if deemed required by the Investigator. Study exit procedures will be performed at the time of withdrawal from the study or as soon as possible thereafter.

### Adverse Events

### Definition of an Adverse Event

An AE is defined as any untoward medical occurrence in a clinical study subject after providing written informed consent for participation in the study that does not necessarily have a causal relationship with the study treatment. An AE can therefore be any unfavorable and unintended sign (including an abnormal laboratory finding), symptom, or disease temporally associated with the use of a medicinal (investigational) product, whether or not it is related to the medicinal (investigational) product. This includes an exacerbation of pre-existing conditions or events, intercurrent illnesses, drug interaction, or the significant worsening of the indication under investigation that is not recorded elsewhere in the CRF under specific efficacy assessments. Anticipated fluctuations of pre-existing conditions that do not represent a clinically significant exacerbation or worsening need not be considered AEs.

### Definition of a Serious Adverse Event

A SAE is any AE that meets any of the following criteria:
Results in death;
Is life-threatening (i.e. in the opinion of the Investigator or designee, the subject is at immediate risk of death from the AE);
Requires inpatient hospitalization or prolongation of existing hospitalization;
Results in persistent or significant disability/incapacity (a substantial disruption of the subject's ability to conduct normal life functions);
Results in a congenital anomaly/birth defect;
Constitutes an important medical event:
   These may not result in death, be life-threatening or require hospitalization, but may be considered serious when they jeopardize the health of the subject and require medical or surgical intervention to prevent one of the outcomes listed.
   Any other event (e.g. allergic bronchospasm requiring intensive treatment in an emergency room or at home, blood dyscrasias, convulsions that do not result in inpatient hospitalization, or the development of drug dependency or drug abuse) thought to be serious (by the Investigator or designee).

### Recording of Adverse Events

AEs will be recorded and evaluated for their seriousness, severity, and relationship to the study medication. AEs will be collected and documented during the course of the study starting from ICF signature. AEs will be followed-up until complete resolution, or until the Investigator judges it to be safe to discontinue follow-up. The relationship to the study medication will be classified according to the clinical site SOPs and the following definitions:

| Severity | Description |
|---|---|
| Mild | Awareness of signs and symptoms, but are easily tolerated; |
| | are of minor irritant type; causing no limitations of usual |
| | activities. Signs or symptoms may require minor action. |
| Moderate | Discomfort severe enough to cause some limitations of |
| | usual activities and may require action. |
| Severe | Incapacitating with inability to carry out usual activities or |
| | significantly affects clinical status, and requires specific |
| | action and/or medical attention. |

### Adverse Events Categories for Determining Relationship to Study Drug

### Related

The AE is maybe, likely, or clearly related to the study drug.

### Unrelated

The AE is clearly unrelated or doubtfully related to the study drug.

### Serious Adverse Events

Information on SAEs will be recorded on the SAE Report Form. Blank copies are included in the study Investigator's file. It is not acceptable for the Investigator to send photocopies of the subject's medical records to the Sponsor company or its representative in lieu of completion of the appropriate AE CRF page or SAE Report Form. However, there may be instances when copies of medical records for certain cases are requested by the Sponsor or its representative. In this instance, all subject identifiers will be redacted on the copies of the medical records before submission to the Sponsor or its representative (this is extremely rare).

The completed SAE Form and SAE cover sheet should be sent via fax or e-mail immediately upon completion to Syneos Health Safety and Pharmacovigilance. If the Investigator does not have all information regarding an SAE, he/she will not wait to receive additional information before completing and sending the form.

Additional relevant information or clinical follow-up should be sent via fax or e-mail to Syneos Health Safety and Pharmacovigilance as soon as it becomes available. The Investigator should follow the subject with the event until resolution or stabilization of the condition. Follow-up reports (as many as required) should be completed and faxed/e-mailed following the same procedure above.

A final report is required once the condition is resolved or stabilized and no more information about the event is expected. The final report should be completed and faxed/e-mailed following the same procedure above.

The Investigator must keep a copy of all documentation related to the event in the clinical site files.

### Serious Adverse Event Reporting to Regulatory Agency(ies)

The Sponsor (or Sponsor's safety representative) is responsible for notifying regulatory agencies of suspected, unexpected, serious adverse reactions (SUSARs) observed during conduct of studies in which the investigational drug is administered.

Notification of fatal or life-threatening SUSARs must be made as soon as possible, but no later than 7 calendar days after becoming aware of the information. Notifications of all other SUSARs that are neither fatal nor life-threatening must be made as soon as possible, but no later than 15 calendar days after becoming aware of the information.

The Sponsor (or Sponsor's safety representative) is responsible to comply with any other applicable regulatory requirement(s) related to the reporting of SAEs to regulatory authority(ies).

### Serious Adverse Event Reporting to the Independent Ethics Committee

In addition to reporting the SAE to the Sponsor (or Sponsor's safety representative), the Investigator must also notify the IEC that approved the study according to their requirements.

It is the responsibility of the site to report as soon as possible, but no later than 7 calendar days after first knowledge by the Investigator, fatal or life-threatening SUSARs occurring at its site to the IEC responsible for the study.

It is the responsibility of the site to report to the IEC all other SUSARs that are neither fatal nor life-threatening, as soon as possible, but no later than 15 calendar days after first knowledge by the Investigator.

Investigator and IEC will also be notified of all significant safety issues (SSIs) that occur during the clinical trial.

Copies of all correspondence relating to reporting of any SAE should be maintained in the study site files and will be checked routinely by the Study Monitor.

### Analytical Methodology

INV-202 and its metabolite(s), if applicable, will be analysed in plasma samples using validated LC-MS/MS methods from subjects who received INV-202 only. Samples from subjects who received placebo will not be analysed.

Once the bioanalytical laboratory confirms receipt of the first shipment, the second set of aliquots may be sent. The samples should be packed on sufficient dry ice to keep them frozen for at least 72 hours.

The bioanalytical work in support of the study will be conducted in compliance with the GCP, using the SOPs in place in the Bioanalytical Division of Syneos Health. These SOPs are in accordance with applicable regulations in the industry: Guidelines on Bioanalytical Method Validation, Good Laboratory Practice (GLP), and Guideline for GCP ICH E6 (R2).

### Safety and Tolerability Evaluations

A complete description of the statistical analyses to be performed on safety and tolerability data will be presented in a SAP.

### Safety Population

The safety population will include all subjects who received at least one dose of the study drug (INV-202 or placebo). The safety population will be used for the summaries of all safety assessments.

### Safety and Tolerability Parameters and Analyses

Safety and tolerability of INV-202 will be evaluated through the assessment of AEs (i.e., seriousness, severity, relationship to the study drug, outcome, duration, and management), vital signs, 12-lead ECG, clinical laboratory parameters, and physical examination.

Demographic parameters will be summarized descriptively.

TEAEs will be tabulated by study treatment and overall for all subjects who were dosed (safety population). Changes from baseline values in vital signs, ECG, and clinical laboratory parameters will be evaluated. Any finding or absence of finding relative to each subject's baseline physical examination will be documented. Any abnormal finding noted after dosing will be documented as an AE if judged as a clinically significant change from baseline. AEs will be coded using the latest version of the Medical Dictionary for Regulatory Activities (MedDRA).

Safety and tolerability data will be reported using descriptive statistics. No inferential statistical analysis of safety data is planned.

### PK and PD Evaluations

A complete description of the statistical analyses to be performed on PK/PD data will be presented in the SAP.

### PK Population

The PK population will include all subjects who received any amount of INV-202 and for whom at least one PK parameter can be adequately characterized.

### PK Parameters

The following PK parameters will be calculated for INV-202 and its major metabolites in plasma concentrations:
1) C_{trough}: concentration reached immediately before the next dose is administered. Will be reported for Days 8, 15 and 22.
2) Mean concentrations: comparison of means will be done based on the values observed in healthy volunteers in the FIH Phase 1 study INV-202-CL-104.
3) Accumulation over the dosing period.

Additional PK analysis may be performed. Upon the Sponsor's request, PK repeats might be performed according to clinical site's SOP.

### PK Statistical Analyses

Descriptive statistics will be provided.

Data will be used to assess differences/similarities with the data obtained from healthy volunteers in the FIH Phase 1 study INV-202-CL-104 and examine potential accumulations. Comparison of concentrations (C_{trough} in subjects with metabolic syndrome vs Cₘᵢₙ observed in healthy volunteers) will be performed to assess differences in PK.

### PD Population

The PD population will include all subjects who received any amount of INV-202 and for whom at least one post-dose PD parameter can be adequately characterized.

### PD Parameters

The following PD parameters will be evaluated:
1) 0-3 hour AUC blood glucose level during OGTT
2) Plasma insulin level—change from baseline to EOS/ET
3) C-peptide level—change from baseline to EOS/ET
4) Plasma lipid profile (HDL, LDL, VLDL, triglycerides)—change from baseline to EOS/ET
5) Adiponectin and leptin—change from baseline to EOS/ET
6) hs-CRP—change from baseline to EOS/ET
7) Weight—change from baseline to EOS/ET
8) Waist circumference—change from baseline to EOT/ET
9) Appetite and Food Intake questionnaire score—change from baseline to EOT/ET
10) Urine albumin to creatinine ratio—change from baseline to EOS/ET

### PD Statistical Analyses

PD analyses will be performed on the PD population.

All PD analyses will be descriptive only and no formal statistical analysis is planned. Descriptive statistics will be used to describe change from baseline in values including glucose AUC during OGTT, fasting insulin level and C-peptide levels, lipids (e.g., triglycerides, HDL), adiponectin and other biomarkers. Exploratory analysis in PD versus AEs may be performed to evaluate the Exposure-Response.

### Compliance

This study will be conducted in compliance with the protocol, GCP, and all applicable regulations, including the Federal Food, Drug and Cosmetic Act, U.S. applicable Code of Federal Regulations (title 21), the Canadian Food and Drugs Act and any IEC requirements relative to clinical studies. The study will also be conducted in compliance with the recommendations laid down in the most recent version of the Declaration of Helsinki, with the exception that registration of such Phase 1 trials in a publicly accessible database is not mandatory. As required by the Canadian regulatory agency, a Clinical Trial Application (CTA) will be submitted before the beginning of the study and a No Objection Letter (NOL) must be received prior to screening.

### Quality Assurance Program

The clinical site has established Quality Control (QC) and Quality Assurance (QA) systems with written SOPs to ensure that the study will be conducted and data will be generated, recorded, and reported in compliance with the protocol, GCP, and applicable regulatory requirements. A rigorous QC program is applied to ensure accuracy of all data and reports. QA oversees a complementary risk-based program of audits to assure compliance with applicable regulations and the clinical site's prescriptive documentation.

### Audits, Inspections and Monitoring

In accordance with the principles of GCP and GLP, the study may be inspected by regulatory authorities, the Sponsor and Syneos Health. The Sponsor is entitled to access information about the status of the study and to review the original documents of the study.

### Confidentiality and Retention of Study Records

This document contains trade secrets and commercial information that is confidential and may not be disclosed to third parties. Persons to whom this study protocol is disclosed must be informed that all the information herein is confidential and may not be further divulged. These restrictions will apply as well to all future communications if deemed privileged or confidential. Publication of the study results may only be allowed with written permission from the Sponsor.

All information on a subject obtained during the conduct of the study will be kept confidential. Subjects will be identified by an anonymized identifier on all samples and study records provided to the Sponsor or designee. In compliance with ICH GCP, the Sponsor's authorized representatives, monitor(s), auditor(s), IEC, and regulatory authority(ies) will be granted direct access to the subject's original trial-related records for verification of clinical trial procedures and/or data, without violating the confidentiality of the subject, to the extent permitted by the applicable laws and regulations. Consent from the subject for disclosure of such information will be obtained in writing in the ICF. In addition, should a subject require medical care or hospitalization during the course of the study, the clinical site may contact the treating physician with the subject's consent, except that consent may not be requested if there is an emergency situation. If the results of the study are published, the subject's identity will remain confidential.

The clinical site will maintain adequate study records for 25 years after completion or termination of study. After this period, the Sponsor will be contacted to determine whether the study records will be forwarded to the Sponsor, destroyed or kept at the clinical site or another facility for a longer period of time at the Sponsor's expense.

### List of Abbreviations

ACEA arachidonyl-2′-chloroethylamide
AE adverse event
ALP alkaline phosphatase
ALT alanine aminotransferase
aPTT activated partial thromboplastin time
AST aspartate aminotransferase
AUC area under the curve
BMI body mass index
BP blood pressure
CB1R cannabinoid-1 receptor
CCKAR cholecystokinin receptor type A
CDER Center for Drug Evaluation and Research
CNS central nervous system
CRF case report form
C-SSRS Columbia Suicidality Severity Rating Scale
CTA Clinical Trial Application
DILI drug induced liver injury
DIO diet-induced obesity
DMP data management plan
ECG electrocardiogram
eGFR estimated glomerular filtration rate
EOS end of study
EOT end of treatment
ET early termination
FDA Food and Drug Administration
FIH First in Human
FSH follicle-stimulating hormone
FU follow-up
GCP Good Clinical Practice
GLP Good Laboratory Practice
GMP Good Manufacturing Practice
GR glucocorticoid receptor
HbA1C hemoglobin A1C
HbsAg Hepatitis B surface antigen
HCV Hepatitis C virus
HDL high-density lipoprotein
HEENT head, eyes, ears, nose, and throat
HEK293 human embryonic kidney 293
hERG human ether-d-go-go-related gene
HIV human immunodeficiency virus
HPBL human peripheral blood lymphocytes
HR heart rate
hs-CRP high sensitivity C-reactive protein
IC₅₀ half-maximal inhibitory concentration
ICF informed consent form
IB investigator's brochure
ICH International Council for Harmonisation of Technical Requirements for Pharmaceuticals for Human Use
IEC Independent Ethics Committee
I_{Kr} rapid delayed rectifier potassium current
INR international normalized ratio
IV intravenous
IVIVC in-vitro in-vivo correlation
KOPR κ opioid receptor
LDL low-density lipoprotein
MAD multiple ascending doses
MDMA 3,4-methylenedioxymethamphetamine
MDRD4 Modification of Diet in Renal Disease (4-variable)
MedDRA Medical Dictionary for Regulatory Activities
MW molecular weight
NAFLD non-alcoholic fatty liver disease
NOAEL no observed adverse effect level
NOL No Objection Letter
OGTT oral glucose tolerance test
OT oral temperature
PCP phencyclidine
PD pharmacodynamic(s)
PK pharmacokinetic(s)
PSA polar surface area
PT prothrombin time
PTT partial thromboplastin time
QA quality assurance
QC quality control
QTcF Fridericia's corrected QT interval
RR respiratory rate
SAD single ascending dose
SAE serious adverse event
SAP statistical analysis plan
SD Standard deviation
SOP standard operation procedure
SSI significant safety issue
SUSAR suspected, unexpected, serious adverse reaction
T4 thyroxine
TBL total bilirubin
TEAE treatment-emergent adverse event
TSH thyroid stimulating hormone
ULN upper limit of normal
V1AR vasopressin type 1A receptor
VAS visual analog scale
VLDL very-low-density lipoprotein

Synopsis of Protocol

| | |
|---|---|
| Study Title: | A Phase 1B study to examine the pharmacokinetic and pharmacodynamic effects of |
| | INV-202 in subjects with metabolic syndrome as defined by hypertriglyceridemia, |
| | abdominal obesity, and impaired glucose tolerance over 28 days |
| Study Drug: | INV-202 |
| Study Phase | Phase 1B - Repeat Dose Study |
| and Type: | |
| Objectives: | To examine the pharmacokinetics (PK) of INV-202 in subjects with |
| | evidence of metabolic syndrome over 28 days. |
| | To examine the pharmacodynamic (PD) effects of INV-202 as |
| | measured by glucose metabolism and lipid profile over 28 days. |
| | To evaluate the safety and tolerability of INV-202 over 28 days. |
| Study | PK Endpoints: |
| Endpoints: | The PK parameters of INV-202 and its major metabolite(s). |
| | Details are available in the PK Section of the protocol and additional details |
| | will be included in the Statistical Analysis Plan (SAP). |
| | PD Endpoints: |
| | 0-3 hour area under the curve (AUC) blood glucose level during the |
| | Oral Glucose Tolerance Test (OGTT) |
| | Change from baseline in: |
| | Plasma insulin level |
| | C-peptide level |
| | Plasma lipid profile (high-density lipoprotein [HDL], low-density lipoprotein [LDL], |
| | very-low-density lipoprotein [VLDL], and triglycerides) |
| | Adiponectin and leptin |
| | High sensitivity C-reactive Protein (hs-CRP) |
| | Weight |
| | Waist circumference |
| | Appetite and Food Intake questionnaire score |
| | Urine albumin to creatinine ratio |
| | Safety Endpoints: |
| | Adverse events (Aes): Frequency, severity, time to onset, duration, and |
| | relatedness to study drug |
| | Serious adverse events (SAEs): Frequency, severity, time to onset, |
| | duration, and relatedness to study drug |
| | Discontinuations due to Aes |
| | Changes in clinical laboratory parameters |
| Study Design: | This will be a single center, Phase 1B, randomized, double-blind, |
| | placebo-controlled, 28-day repeat dose study. |
| | The study will include subjects with evidence of metabolic syndrome and |
| | glucose intolerance that will be randomized 1:1 to receive either INV-202 or |
| | placebo. 20 subjects will receive 25 mg of INV-202 and 20 subjects will |
| | receive a matching placebo daily for 28 consecutive days, for a total of 40 subjects. |
| Subjects: | This study plans to enroll up to 40 subjects with metabolic syndrome (as defined by |
| | hypertriglyceridemia, abdominal obesity, and impaired glucose tolerance), and |
| | without other significant and uncontrolled or unstable medical conditions. |
| | The subjects will be males or females, ≥18 and ≤65 years of age. |
| | The subjects must have evidence of glucose intolerance (OGTT or |
| | hemoglobin A1C [HbA1C] ≥5.7% but ≤6.4%), elevated fasting triglyceride |
| | levels (>1.5 mmol/L for males and females), and a waist circumference of at |
| | least 88 cm for females and 102 cm for males. |
| Screening | Demographic data, medical and medication histories, complete physical examination, |
| Procedures: | body measurements, Columbia Suicidality Severity Rating Scale (C-SSRS), OGTT, |
| | vital signs (blood pressure [BP], heart rate [HR], respiratory rate [RR], and oral |
| | temperature [OT]), 12-lead electrocardiogram (ECG), hematology, biochemistry, |
| | coagulation, endocrinology (only follicle-stimulating hormone [FSH]), serology |
| | (human immunodeficiency virus [HIV] antigen and antibody, hepatitis B surface |
| | antigen [HbsAg], and hepatitis C virus [HCV] antibody), urinalysis, serum pregnancy |
| | test, alcohol breath test, and urine drug screen. |
| Inclusion | Subjects must meet all of the following criteria to be included in the study: |
| Criteria: | Provision of a signed and dated informed consent form (ICF). |
| | Willing and able to comply with all study procedures for the duration of the study. |
| | Male or female, ≥18 and ≤65 years of age. |
| | Waist circumference ≥88 cm for female subjects or ≥102 cm for male subjects. |
| | Fasting triglyceride >1.5 mmol/L for males and females. |
| | An OGTT indicating impaired glucose tolerance as indicated by a 2-hour |
| | value >140 mg/dl or any value >200 mg/dl at any time point or a |
| | HbA1C level ≥5.7% but ≤6.4%. |
| | Females of childbearing potential who are sexually active with a non-sterile male |
| | partner (sterile male partners are defined as men vasectomized at least 6 months prior |
| | to the first study drug administration) must be willing to use one of the following |
| | acceptable contraceptive methods throughout the study and for at least 30 days after |
| | the last study drug administration: |
| | a) Simultaneous use of hormonal contraceptives, started at least 4 weeks prior to |
| | study drug administration and must agree to use the same hormonal contraceptive |
| | throughout the study, and condom for the male partner; |
| | b) Simultaneous use of intra-uterine contraceptive device, placed at least 4 weeks |
| | prior to study drug administration, and condom for the male partner; |
| | c) Simultaneous use of diaphragm or cervical cap with intravaginally |
| | applied spermicide and male condom for the male partner, started at |
| | least 21 days prior to study drug administration. |
| | Females of non-childbearing potential must be: |
| | d) Post-menopausal (absence of menses for at least 12 months prior to the first study |
| | drug administration) with confirmation of the post-menopausal status by documented |
| | FSH level ≥40 mIU/mL; or |
| | e) Surgically sterile (complete hysterectomy, bilateral oophorectomy or tubal ligation |
| | at least 6 months prior to the first study drug administration). |
| | Male subjects who are not vasectomized for at least 6 months, and who are sexually |
| | active with a female partner of childbearing potential (childbearing potential females |
| | are defined as women that are neither post-menopausal nor surgically sterile) must be |
| | willing to use one of the following acceptable contraceptive methods from the first |
| | study drug administration until at least 90 days after the last study drug administration: |
| | f) Simultaneous use of a male condom and, for the female partner, |
| | hormonal contraceptives used since at least 4 weeks or intra-uterine |
| | contraceptive device placed since at least 4 weeks; |
| | g) Simultaneous use of a male condom and, for the female partner, a |
| | diaphragm or cervical cap with intravaginally applied spermicide. |
| | Male subjects (including men who have had a vasectomy) with a pregnant |
| | partner must agree to use a condom from the first study drug |
| | administration and for 90 days after the last study drug administration. |
| | Male subjects must be willing not to donate sperm for 90 days following the |
| | last study drug administration. |
| Exclusion | Subjects to whom any of the following applies will be excluded from the study: |
| Criteria: | 1) Female who is lactating at screening. |
| | 2) Female who is pregnant according to a pregnancy test at screening or |
| | prior to study drug administration. |
| | 3) History of significant hypersensitivity to the study drug or excipients of |
| | the study drug. |
| | 4) History of severe hypersensitivity reactions, such as anaphylaxis. |
| | 5) History of rare hereditary problems of galactose and/or lactose |
| | intolerance, lactase deficiency or glucose-galactose malabsorption. |
| | 6) Positive screening results to HIV antigen and antibody, HbsAg or HCV tests. |
| | 7) Poses a significant suicidal risk as defined by C-SSRS score >Type 1 |
| | ideation at screening. |
| | 8) Any clinically significant illness in the 28 days prior to study drug administration. |
| | 9) Presence or history of significant gastrointestinal, liver or kidney |
| | disease, or surgery that may affect drug bioavailability. |
| | 10) History of significant and uncontrolled or unstable cardiovascular, |
| | pulmonary, hematologic, neurological, psychiatric, endocrine, |
| | immunologic or dermatologic disease. |
| | 11) History of seizures (epilepsy) of any kind. |
| | 12) History of cranial surgery. |
| | 13) Presence of clinically significant ECG abnormalities at the screening |
| | visit, as defined by medical judgment (maximum Fridericia's corrected |
| | QT interval [QTcF] 450 msec). |
| | 14) Any other clinically significant abnormalities in laboratory test results at |
| | screening that would, in the opinion of an investigator, increase the subject's risk |
| | of participation, jeopardize complete participation in the study, or compromise |
| | interpretation of study data. Mild elevations in aspartate aminotransferase |
| | (AST)/alanine aminotransferase (ALT) as may be seen in non-alcoholic fatty liver |
| | disease (NAFLD) are not exclusionary. However, in these subjects, please follow the |
| | hepatic safety section. |
| | 15) New prescription medication or changes to medication regimen within 90 days |
| | prior to the first dose. (i.e., stable doses of antihypertensives etc. are allowed). |
| | 16) Use of the following medications for the timeframes specified below, with the |
| | exception of medications exempted by the Investigator on a case-by-case basis |
| | because they are judged unlikely to affect the PK profile of the study drug or subject |
| | safety (e.g., topical drug products without significant systemic absorption): |
| | a) Any vaccination, including COVID-19 vaccine, within 14 days |
| | prior to the first dose; |
| | b) Depot injection or implant of any drug within 3 months prior to the first dose; |
| | c) Any drugs known to induce or inhibit hepatic drug metabolism |
| | within 30 days prior to the first dose. |
| | d) Any drugs for the treatment of diabetes within 30 days prior to the first dose. |
| | e) Any drugs prohibited by the Investigator on a case-by-case basis because they are |
| | judged likely to affect the PD profile of the study drug or subject safety, within at |
| | least 5 times the half-life of the drug and a minimum of 30 days prior to the first dose. |
| | 17) Positive urine drug screen or alcohol breath test at screening. |
| | 18) History of significant alcohol abuse within 1 year prior to screening or regular use |
| | of alcohol within 6 months prior to the screening visit (more than 14 units of alcohol |
| | per week [1 unit = 150 mL of wine, 360 mL of beer, or 45 mL of 40% alcohol]). |
| | 19) History of significant drug abuse within 1 year prior to screening, use of |
| | soft drugs within 3 months prior to screening, marijuana within 1 month |
| | prior to screening, or hard drugs (such as cocaine, phencyclidine [PCP], |
| | crack, opioid derivatives including heroin, and amphetamine |
| | derivatives) within 1 year prior to screening. |
| | 20) Use of any cannabinoid containing product, including cannabis, within |
| | 1 month prior to screening, by any route (e.g., oral, inhaled, topical). |
| | 21) Participation in a clinical research study involving the administration of an |
| | investigational or marketed drug or device within 30 days (or a minimum of 5 |
| | half-lives, whichever is longer) prior to the first dose, administration of a biological |
| | product in the context of a clinical research study within 90 days prior to the first |
| | dose, or concomitant participation in an investigational study involving no drug or |
| | device administration. |
| | 22) Donation of plasma within 7 days prior to dosing. Donation or loss of blood |
| | (excluding volume drawn at screening) of 50 mL to 499 mL of blood within 30 days, |
| | or more than 499 mL within 56 days prior to the first dose. |
| | 23) Any reason, which in the opinion of the Investigator, would prevent the |
| | subject from participating in the study. |
| Study Visits: | Subjects will undergo 8 study visits: |
| | A screening visit within 35 days of randomization; |
| | A first baseline visit on Day −1; |
| | A second baseline visit on Day 1; |
| | 3 weekly visits during the treatment period on Days 8, 15, and 22; |
| | An end of treatment (EOT) visit on Day 29 (+2 days); and |
| | An end of study (EOS) visit on Day 35 (+2 days). |
| Study Drug and | Test product: INV-202: 25 mg tablets. |
| Dosage Form: | Placebo: Tablets identical in appearance color, shape and size to the active |
| | INV-202 tablets. |
| Study Drug | Subjects will receive a daily oral dose of INV-202 25 mg or a matching |
| Administration: | placebo for 28 consecutive days. |
| | At the baseline visit and follow-up visits on Days 8, 15, and 22, no food will be |
| | allowed from at least 8 hours prior to arrival to the clinic until the completion of all |
| | scheduled assessments. After completion of the assessments, food will be provided, |
| | and the daily dose will be taken at the site with food. For all other doses, subjects |
| | will take the study drug independently and will be instructed to self-administer doses |
| | of the study drug once daily with food. |
| Study | Subjects will be asked to refrain from using products that may potentially affect their |
| Restrictions: | safety and/or the PK profile of the study drug. Main study restrictions include: |
| | new prescription medication or changes to medication regimen from 90 days prior to |
| | the first dose until after the last PK blood sample collection of the study; |
| | any vaccination, including COVID-19 vaccine, from 14 days prior to the |
| | first dose until after the last PK blood sample collection of the study; |
| | food or beverages containing grapefruit, starfruit, pomegranate, pineapple, or pomelo |
| | from 7 days pre-dose until after the last PK blood sample collection of the study; |
| | alcohol-based products from 8 hours prior to each visit. Subjects will be |
| | asked to limit alcohol intake to a moderate level (<2 drinks per day) |
| | from Day −1 until study completion. |
| | An OGTT will be performed at screening, on Day −1, and on Day 29 (+2 days). |
| | Since activity can interfere with test results, subjects will be required to remain |
| | seated, unless medically necessary or required by procedures, from the time the |
| | glucose load is administered until the last blood draw (2-hours post-intake at |
| | screening and 3 hours post-intake at other visits) is completed. |
| Blood Sample | Approximately 6 blood samples will be collected for PK/PD assessments at the |
| Collection for | following time points: pre-dose on Days 1, 8, 15, and 22, on Day 29 (+2 days), and at |
| PK/PD Analysis: | the Day 35 (+2 days) follow-up (FU)/EOS/early termination (ET) visit. |
| Hepatic Safety | The following instructions are to be followed if liver function markers and |
| Precautions: | clinical presentation suggest the onset of hepatic impairment during the |
| | study. The hepatic safety of all randomized subjects will be assessed through |
| | the use of the following algorithm based on the FDA Drug Induced Liver |
| | Injury (DILI) management guidelines (guidelines (Center for Drug |
| | Evaluation and Research (CDER), FDA. Guidance for Industry: Drug-Induced |
| | Liver Injury: Premarketing Clinical Evaluation. July 2009)) |
| | An increase of serum ALT or AST > 3 × ULN should be followed by repeat |
| | testing within 48 to 72 hours of all four of the usual serum measures (ALT, |
| | AST, alkaline phosphatase [ALP], and total bilirubin [TBL]) to confirm the |
| | abnormalities and to determine if they are increasing or decreasing. There |
| | also should be inquiry made about symptoms. |
| | The need for prompt repeat testing is especially great if ALT or AST is |
| | much greater than 3 × ULN and/or TBL is greater than 2 × ULN. |
| | If symptoms persist or repeat testing shows ALT or AST > 3 × ULN for subjects with |
| | normal baseline measures or 2-fold increases above baseline values for subjects with |
| | elevated values before drug exposure, it is appropriate to initiate close observation to |
| | determine whether the abnormalities are improving or worsening. Close observation |
| | includes repeat testing two or three times weekly, detailed medical and medication |
| | history, ruling out other causes, etc. If close monitoring is not possible, the drug |
| | should be discontinued. Moreover, INV-202 should be discontinued if: |
| | ALT or AST > 5 × ULN |
| | ALT or AST > 3 × ULN and (TBL > 2 × ULN or international |
| | normalized ratio [INR] > 1.5) |
| | ALT or AST > 3 × ULN with the appearance of fatigue, nausea, vomiting, |
| | right upper quadrant pain or tenderness, fever, rash, and/or eosinophilia (>5%). |
| On-Study | Medical surveillance: Throughout the study, subjects will be monitored by |
| Safety | the clinical staff for Aes. The Investigator or designee will be on site for the |
| Monitoring: | first study drug administration. |
| | Brief physical examination: Pre-dose on Days 1, 8, 15, and 22, and on Day |
| | 29 (+2 days). |
| | Body measurements (weight and waist circumference only): Pre-dose on |
| | Days 1, 8, 15, and 22, and on Day 29 (+2 days). |
| | Vital signs: Pre-dose on Days 1, 8, 15, and 22, and on Day 29 (+2 days). |
| | 12-lead ECG: Pre-dose on Day 1 and on Day 29 (+2 days). |
| | Laboratory assessments: |
| | Urine pregnancy test: Pre-dose on Day 1. |
| | Hematology, biochemistry, coagulation, endocrinology, and urinalysis: |
| | Pre-dose on Days 1, 8, 15, and 22, and on Day 29 (+2 days). |
| | OGTT: On Day −1 and Day 29 (+2 days). |
| | Questionnaires: |
| | C-SSRS: On Day −1 and Day 29 (+2 days). |
| | Appetite and Food intake questionnaire: Pre-dose on Days 1, 8, 15, and 22, |
| | and on Day 29 (+2 days). |
| | Throughout the treatment period, subjects will record the time of dosing, any |
| | observations regarding the taste of the tablets, as well as any Aes that they |
| | may experience, in a subject diary card. |
| | Additional safety measurements may be performed at the discretion of the |
| | Investigator for reasons related to subject safety. |
| FU | A FU visit will occur on Day 35 (+2 days), approximately 168 hours |
| Visit/EOS/ET: | following the last study drug administration. |
| | EOS procedures are scheduled to be performed at the FU visit and will consist |
| | of the following assessments: complete physical examination, C-SSRS, vital |
| | signs, 12-lead ECG, hematology, biochemistry, coagulation, endocrinology, |
| | urinalysis, urine pregnancy test, PK/PD blood sampling and AE monitoring. |
| | These procedures will be performed within 14 days after the last |
| | participation of the subject in the study in case of ET. Subjects who |
| | discontinue from the study prematurely will undergo an ET visit. |
| Analytical | The Bioanalytical Division of Syneos Health will analyse INV-202 and its |
| Method: | major metabolite(s) in plasma samples using validated methods. Only |
| | INV-202 treated subjects will be analysed. |
| Safety and | Safety and tolerability to INV-202 will be evaluated through the assessment |
| Tolerability | of Aes (i.e., seriousness, severity, relationship to the study medication, |
| Parameters: | outcome, duration, and management), vital signs, ECGs, clinical laboratory |
| | parameters, and physical examination. |
| PK Parameters: | The following parameters will be calculated for INV-202 and its major |
| | metabolite(s) in plasma concentrations: C_{trough}, mean concentrations and |
| | accumulation over the dosing period. |
| PD Parameters: | The following PD parameters will be calculated: 0-3 hour AUC blood |
| | glucose level during OGTT. Changes from baseline in: plasma insulin level, |
| | C-peptide level, plasma lipid profile (HDL, LDL, VLDL, and triglycerides), |
| | adiponectin and leptin, hs-CRP, weight, waist circumference, Appetite and |
| | Food Intake questionnaire score and urine albumin to creatinine ratio. |
| Statistical | A complete description of the statistical analyses to be performed on safety, |
| Analyses: | tolerability, PK and PD data will be presented in a SAP. Samples from |
| | subjects who received placebo will be excluded from PK/PD analysis. |
| | PK analysis: |
| | Descriptive statistics will be provided. Comparison of concentrations (C_{trough} in |
| | subjects with metabolic syndrome vs Cₘᵢₙ observed in healthy volunteers from the |
| | First in Human (FIH) Phase 1 study INV-202-CL-104) to assess differences in PK. |
| | PD Analysis: |
| | All PD analyses will be descriptive only and no formal statistical analysis is planned. |
| | Descriptive statistics will be used to describe change from baseline in values |
| | including glucose AUC during OGTT, fasting insulin level and C-peptide levels, |
| | lipids (e.g., triglycerides, HDL), adiponectin and other biomarkers._Exploratory |
| | analysis in PD versus Aes may be performed to evaluate the Exposure-Response. |
| | Safety and tolerability analysis: |
| | Safety analyses will be performed on the safety population. Treatment-emergent Aes |
| | (TEAEs) will be tabulated by study treatment. Changes from baseline values in vital |
| | signs, ECG, and clinical laboratory parameters will be evaluated. Safety parameters, |
| | including Aes, SAEs, labs, vital signs, and ECG findings will be assessed and |
| | reported using descriptive statistics. |

Schedule of Events

| | | | | Weekly Visits | End of | Day 35 (+2) |
|---|---|---|---|---|---|---|
| | Screening | Baseline | | Days 8, 15 & | Treatment | FU Visit/ |
| PROCEDURE | Day −35 to −2 | Day −1 | Day 1 | 22 | Day 29 (+2) | EOS/ET¹¹ |
| 1. Informed Consent | X | | | | | |
| 2. Demographic Data | X | | | | | |
| 3. Medical and Medication | X | | X | | | |
| Histories | | | | | | |
| 4. Review and Monitoring | X | X | X | X | X | X |
| of AEs and Concomitant | | | | | | |
| Medications | | | | | | |
| 5. Physical Examination¹ | X | | X | X | X | X |
| 6. Body Measurements | X | | X | X | X | |
| (height, weight, BMI, waist | | | | | | |
| circumference)² | | | | | | |
| 7. C-SSRS Scale³ | X | X | | | X | X |
| 8. Vital Signs (BP, HR, RR, | X | | X | X | X | X |
| OT) | | | | | | |
| 9. 12-lead ECG | X | | X | | X | X |
| 10. OGTT^{4,5} | X | X | | | X | |
| 11. Hematology⁵ | X | | X | X | X | X |
| 12. Biochemistry⁵ | X | | X | X | X | X |
| 13. Coagulation⁵ | X | | X | X | X | X |
| Endocrinology^{5,6} | X | | X | X | X | X |
| 14. Serology (HIV, Hepatitis | X | | | | | |
| B and C)⁵ | | | | | | |
| 15. Serum Pregnancy Test | X | | | | | |
| 16. Urine Pregnancy Test⁷ | | | X | | | X |
| 17. Urinalysis⁵ | X | | X | X | X | X |
| 18. Appetite and Food | | | X | X | X | |
| Intake Questionnaire | | | | | | |
| 19. Diary Card Review⁸ | | | | X | X | |
| 20. Urine Drug Screen | X | | | | | |
| 21. Alcohol Breath Test | X | | | | | |
| 22. Drug Administration⁹ | | | X | X | | |
| 23. PK Blood Samples¹⁰ | | | X | X | X | X |
| 24. PD Blood Samples¹⁰ | | | X | X | X | X |

¹A complete physical examination will be performed at screening and Day 35 (+2 days) FU Visit/EOS/ET. A brief physical examination will be performed on Days 1, 8, 15, 22, and 29. Other physical examinations will be carried out at the discretion of the Investigator.
²Height will only be measured at screening. Weight and waist circumference will be measured pre-dose on Days 1, 8, 15, 22 and Day 29 (+2 days). Weight and waist circumference will be measured at all timepoints in triplicate using a standard measuring method. Body mass index (BMI) will be calculated as needed.
³If the screening C-SSRS was performed more than 21 days prior to Day −1, a repeat C-SSRS should be performed on Day −1. Otherwise, the screening C-SSRS may be used as the baseline assessment.
⁴The OGTT will not need to be performed at screening if there is evidence of HbA1C ≥5.7% but ≤6.4% obtained within the last 3 months. At screening (if applicable), on Day −1 and on Day 29 (+2 days), subjects will undergo an OGTT consisting of a 75 g glucose load with blood draws pre-load (if applicable) and at 30 minutes, 1 hour, 2 hours, and 3 hours (if applicable) post-intake.
⁵Lab tests will be performed following an 8-hour fast.
⁶FSH level will only be measured at screening to confirm post-menopausal status. C-peptide, insulin, thyroid stimulating hormone (TSH), and thyroxine (T4) will be measured pre-dose on Days 1, 8, 15, and 22, on Day 29 (+2 days), and at the FU Visit/EOS/ET on Day 35 (+2 days).
⁷A urine pregnancy test that yields a positive result will be confirmed by a serum pregnancy test.
⁸Throughout the treatment period, subjects will record the time of dosing, any observations regarding the taste of the tablets, as well as any AEs that they may experience, in a subject diary card. Diary cards will be collected and reviewed on Days 8, 15, 22 and 29 (+2 days).
⁹Subjects will receive oral doses of INV-202 or matching placebo once daily from Day 1 to Day 28. Each study drug administration will be separated by approximately 24 hours and should be done at approximately the same time every day.
¹⁰PK/PD blood samples will be collected on Days 1, 8, 15, 22 pre-dose, on Day 29 (+2 days) and on Day 35 (+2 days) FU Visit/EOS/ET.

EOS procedures are scheduled to be performed at the FU on Day 35 (+2 days) or within 14 days after the last participation of the subject in the study in case of ET. Subjects who discontinue from the study prematurely will undergo an ET visit

### Study Results

### I. Change in Weight, Waist Circumference, BMI

INV-202 was shown to induce weight loss, to reduce waist circumferences and BMI as shown in FIG. 1 and Table 1 below. Every single patient on INV-202 lost weight. The maximum weight loss by percentage after 28 days of treatment was 6% and the lowest was 0.1%. A statistically significant changes in weight was noted at the first clinic visit (day 8) which was an average of 1.4% weight loss.

TABLE 1

| Change from | | | | | |
|---|---|---|---|---|---|
| Baseline (SD) | INV-202 | | Placebo | | P value |
| Weight (kg) | −3.50 | (1.42) | 0.55 | (0.80) | P < 0.01 |
| Weight (%) | −3.33% | (1.36) | 0.50% | (0.73) | P < 0.01 |
| Waist (cm) | −1.91 | (2.2) | 0.02 | (3.0) | P = 0.036 |

| | | | |
|---|---|---|---|
| BMI (kg/m²) | −1.20 | 0.21 | P < 0.01 |

### II. Lipids and Glucose

INV-202's effect on lipids and glucose is shown in Table 2 below:

TABLE 2
Change from baseline

| (SD) | INV-202 | | Placebo | | P value |
|---|---|---|---|---|---|
| Triglycerides (mmol/L) | −0.179 | (0.54) | 0.095 | (0.36)) | P = 0.08 |
| Triglycerides (%) | −7.9% | ( | 4.4% | | |
| HDL (mmol/L) | 0.0015 | (0.10) | −0.02 | (0.08) | P = 0.48 |
| HDL (%) | 0.13% | | −1.7% | | |
| LDL (mmol/L) | −0.50 | (0.59) | 0.02 | (0.30) | P = 0.004 |
| VLDL (mmol/L) | −0.08 | (0.25) | 0.04 | (0.17) | p = 0.09 |
| Total Chol. (mmol/L) | −0.56 | (0.60) | 0.04 | (0.39) | p = 0.002 |
| OGTT (hr*mmol/L) | 2.24 | (4.76) | 3.46 | (4.00) | p = 0.192 |
| HgbA1C (%) | −0.005 | (1.8) | 0.065 | (1.5) | p = 0.058 |
| C-peptide (nmol/L) | 0.036 | (0.16) | 0.054 | (0.19) | p = 0.71 |
| Insulin (pmol/L) | −5.0 | (46) | −1.2 | (60) | p = 0.41 |
| hs-CRP (mg/L) | −1.6 | (2.5) | 0.6 | (4.1) | p = 0.054 |

### III. Endocrine and Renal Lab Data

INV-202 effect on endocrine and renal markers is shown below.

TABLE 3

| (SD) | INV-202 | | Placebo | | P value |
|---|---|---|---|---|---|
| T4 (pmol/L) | 0.516 | | −0.782 | | P = 0.02 |
| TSH (mIU/L) | −0.014 | | 0.709 | | P = 0.008 |
| Adiponectin (mg/L) | 0.15 | (1.6) | −0.65 | (1.6) | p = 0.13 |
| Leptin (ug/L) | −2.64 | (4.5) | −1.66 | (7.1) | p = 0.35 |

| | | | |
|---|---|---|---|
| Leptin (%) | −18% | | |
| eGFR (CKD-EPI 2021) | 3.23 | −0.75 | P = 0.07 |
| mL/min/1.73 m2 | | | |

### IV. C-SSRS Summary

As shown below:
No subject, before or after treatment, had any active, non-specific suicidal thoughts.
No subject, before or after treatment, had an actual, interrupted, planned or aborted suicide attempt.

TABLE 4

| | Total | | INV-202 | | Placebo | |
|---|---|---|---|---|---|---|
| | Baseline | EOT | Baseline | EOT | Baseline | EOT |
| Wished to be dead | 0 | 0 | 0 | 0 | 0 | 0 |
| Non-specific active | 0 | 0 | 0 | 0 | 0 | 0 |
| suicidal thoughts | | | | | | |
| Suicidal behaviour | 0 | 0 | 0 | 0 | 0 | 0 |

### V. Liver Safety

All liver chemistry were within normal limits for all patients throughout the study. There were no significant changes for bilirubin (total, direct, indirect) or transaminases (AST and ALT).

### VI. Vital Signs

TABLE 5

| Change from baseline | INV-202 | Placebo | p value |
|---|---|---|---|
| Systolic Blood Pressure | −4.65 | 1.64 | P = 0.21 |
| (SBP) (mmHg) | | | |
| Diastolic Blood Pressure | −0.25 | −0.94 | P = 0.73 |
| (DBP) (mmHg) | | | |
| Heart Rate (bpm) | 2.3 | −0.05 | P = 0.41 |
| Temp (° C.) | −0.045 | −0.018 | P = 0.70 |

Although the invention has been illustrated and described with respect to one or more implementations, equivalent alterations and modifications will occur to others skilled in the art upon the reading and understanding of this specification. In addition, while a particular feature of the invention may have been disclosed with respect to only one of several implementations, such feature may be combined with one or more other features of the other implementations as may be desired and advantageous for any given or particular application.

Accordingly, it is understood that the examples and embodiments described herein are for illustrative purposes only and that various modifications or changes in light thereof will be suggested to persons skilled in the art and are to be included within the spirit and purview of this application and scope of the appended claims. Any publication, document, patent, patent application or publication referred to herein should be construed as incorporated by reference each in their entirety for all purposes.

1.-27. (canceled)

28. A method for reducing body weight and triglycerides in a human subject determined as having abdominal obesity characterized by a waist circumference ≥88 cm for female subjects or ≥102 cm for male subjects, wherein said reduction is relative to baseline level before treatment said method comprising orally administering a compound of Formula I or a pharmaceutically acceptable salt thereof: 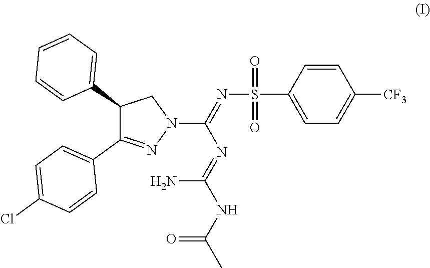 [SMILES: CC(=O)N/C(N)=N/C(=N\S(=O)(=O)c1ccc(C(F)(F)F)cc1)N1C[C@H](c2ccccc2)C(c2ccc(Cl)cc2)=N1]
to the human subject for a period of at least 28 days.

29. A method for reducing body weight, triglycerides and glucose intolerance in a human subject determined as having abdominal obesity characterized by a waist circumference ≥88 cm for female subjects or ≥102 cm for male subjects, wherein said reduction is relative to baseline level before treatment said method comprising orally administering a compound of Formula I or a pharmaceutically acceptable salt thereof: 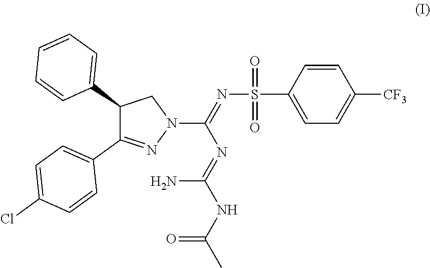 [SMILES: CC(=O)N/C(N)=N/C(=N\S(=O)(=O)c1ccc(C(F)(F)F)cc1)N1C[C@H](c2ccccc2)C(c2ccc(Cl)cc2)=N1]
to the human subject for a period of at least 28 days.

30. The method of claim 28 wherein the subject presents one or more of the following:
fasting triglycerides >1.5 mmol/L for males and females and/or an OGTT indicating impaired glucose tolerance as indicated by a 2-hour value >140 mg/dl or any value >200 mg/dl at any time point or a HbA1C level ≥5.7% but ≤6.4%.

31. The method of claim 28 wherein the subject exhibits no significant changes relative to baseline for bilirubin, AST and/or ALT after 28 days of treatment with the compound of formula I.

32. The method of claim 28 wherein the subject exhibits a body weight reduction relative to baseline between about 0.1 and about 7%, between about 1 and about 6%, between about 2 and about 5%, between about 3 and about 5%, about 4%, about 3.5%, about 3% or about 2.5% after 28 days of treatment with the compound of formula I.

33. The method of claim 28 wherein the subject exhibits a triglycerides reduction relative to baseline between about 1 and about 10%, between about 2 and about 9%, between about 3 and about 8%, about 8%, about 7%, about 6% or about 5% after 28 days of treatment with the compound of formula I.

34. The method of claim 28 wherein the subject exhibits a change in leptin levels relative to baseline of about between 10% and about 20%, after 28 days of treatment with the compound of formula I.

35. The method of claim 28 wherein the subject exhibits a reduction in LDL level relative to baseline of at least about 0.1 mmol/L to about 1 mmol/L after 28 days of treatment with the compound of formula I.

36. The method of claim 28 wherein the subject exhibits a reduction in VLDL level relative to baseline of at least about 0.03 mmol/L to about 1 mmol/L after 28 days of treatment with the compound of formula I.

37. The method of claim 28 wherein the subject exhibits a reduction in total cholesterol levels relative to baseline of at least about 0.1 mmol/L to about 1 mmol/L after 28 days of treatment with the compound of formula I.

38. The method of claim 28 wherein the compound of Formula I is for oral administration to the subject at a dose of 25 mg or more.

39. The method of claim 28 wherein the compound of Formula I is for daily oral administration to the subject at a dose of 25 mg or more.

40. The method of claim 28 wherein the compound of Formula I is for daily oral administration to the subject at a dose of about 25 mg.

41. The method of claim 28 wherein the compound of Formula I is for daily oral administration to the subject at a dose of less than 25 mg.

42. The method of claim 29 wherein the subject presents one or more of the following:
fasting triglycerides >1.5 mmol/L for males and females and/or an OGTT indicating impaired glucose tolerance as indicated by a 2-hour value >140 mg/dl or any value >200 mg/dl at any time point or a HbA1C level ≥5.7% but ≤6.4%.

43. The method of claim 29 wherein the subject exhibits no significant changes relative to baseline for bilirubin, AST and/or ALT after 28 days of treatment with the compound of formula I.

44. The method of claim 29 wherein the subject exhibits a body weight reduction relative to baseline between about 0.1 and about 7%, between about 1 and about 6%, between about 2 and about 5%, between about 3 and about 5%, about 4%, about 3.5%, about 3% or about 2.5% after 28 days of treatment with the compound of formula I.

45. The method of claim 29 wherein the subject exhibits a triglycerides reduction relative to baseline between about 1 and about 10%, between about 2 and about 9%, between about 3 and about 8%, about 8%, about 7%, about 6% or about 5% after 28 days of treatment with the compound of formula I.

46. The method of claim 29 wherein the subject exhibits a change in leptin levels relative to baseline of about between 10% and about 20%, after 28 days of treatment with the compound of formula I.

47. The method of claim 29 wherein the subject exhibits a reduction in LDL level relative to baseline of at least about 0.1 mmol/L to about 1 mmol/L after 28 days of treatment with the compound of formula I.

48. The method of claim 29 wherein the subject exhibits a reduction in VLDL level relative to baseline of at least about 0.03 mmol/L to about 1 mmol/L after 28 days of treatment with the compound of formula I.

49. The method of claim 29 wherein the subject exhibits a reduction in total cholesterol levels relative to baseline of at least about 0.1 mmol/L to about 1 mmol/L after 28 days of treatment with the compound of formula I.

50. The method of claim 28 wherein the subject exhibits no significant changes in subjective appetite sensations during treatment as determined by an appetite and Food Intake Questionnaire.

51. The method of claim 29 wherein the subject exhibits no significant changes in subjective appetite sensations during treatment as determined by an appetite and Food Intake Questionnaire.

52. The method of claim 29 wherein the compound of Formula I is for oral administration to the subject at a dose of 25 mg or more.

53. The method of claim 29 wherein the compound of Formula I is for daily oral administration to the subject at a dose of 25 mg or more.

54. The method of claim 29 wherein the compound of Formula I is for daily oral administration to the subject at a dose of about 25 mg.

55. The method of claim 29 wherein the compound of Formula I is for daily oral administration to the subject at a dose of less than 25 mg.
